(19) United States
(12) Patent Application Publication
(10) Pub. No.: US 2014/0094493 A1
(43) Pub. Date: Apr. 3, 2014

(54) Novel Ether Linked Compounds and Improved Treatments for Cardiac and Cardiovascular Disease

(71) Applicants: Jillian G. Baker, Nottingham (GB); Peter M. Fischer, Nottingham (GB); Christophe Fromont, Nottingham (GB); Sheila M. Gardiner, Nottingham (GB); Stephen J. Hill, Nottingham (GB); Gopal Jadhav, Nottingham (GB); Barrie Kellam, Nottingham (GB); Shailesh Mistry, Nottingham (GB); Jeanette Woolard, Nottingham (GB)

(72) Inventors: Jillian G. Baker, Nottingham (GB); Peter M. Fischer, Nottingham (GB); Christophe Fromont, Nottingham (GB); Sheila M. Gardiner, Nottingham (GB); Stephen J. Hill, Nottingham (GB); Gopal Jadhav, Nottingham (GB); Barrie Kellam, Nottingham (GB); Shailesh Mistry, Nottingham (GB); Jeanette Woolard, Nottingham (GB)

(21) Appl. No.: 13/983,462

(22) PCT Filed: Feb. 6, 2012
(86) PCT No.: PCT/GB2012/050246

§ 371 Date: Dec. 2, 2013

(30) Foreign Application Priority Data
Feb. 4, 2011 (GB) 1101959.3

(51) Int. Cl. C07D 405/14; C07D 319/20; C07D 211/38

(52) U.S. Cl. CPC C07D 405/14; C07D 211/38; C07D 319/20
USPC 514/321; 548/454; 514/414; 546/234; 514/331; 546/197; 549/366; 514/452

(57) ABSTRACT

A compound of Formula (I), and its pharmaceutically acceptable salt or salts and physiologically hydrolysable derivatives in free form or salt form:

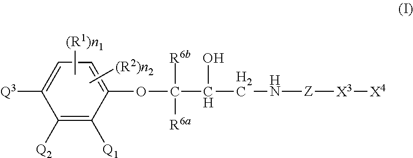

wherein
R¹ is independently selected from F, Cl, Br, CN, NH₂, OH, CHO, COOH, oxo, C₁₋₄alkyl, C₁₋₄alkoxy, CONH₂ (optionally mono- or di-substituted by C₁₋₄alkyl) and SO₂NH₂,
R² is independently selected from C₁₋₆allkyl substituted by R³ wherein the C₁₋₆alkyl chain optionally comprises one or two heteroatoms select from O;
R³ is selected from aryl, C₃₋₆cycloalkyl, C₃₋₆heterocyclyl and C₃₋₆heteroaryl, wherein the heterocyclyl and heteroaryl rings are nitrogen containing;
   and wherein R³ is optonally substituted by one or more groups selected from R¹;
n1 is zero or an integer from 1 to 2;
n2 is an integer from 1 to 2;
and the sum of n1 and 2 is less than or equal to 2;
R⁵ is selected from any group defined for R¹ and R²;
R^{6a} and R^{6b} are independently selected from H or C₁₋₄alkyl;
R⁷ is independently selected from F, Cl, Br, CN, NH₂, OH, CHO, COOH, oxo, C₁₋₄alkyl, C₁₋₄alkoxy, CONH₂ (optionally mono- or di-substituted by C₁₋₄alkyl) and SO₂NH₂,
Q¹, Q² and Q³ are independently selected from H or any group defined for R¹ and R²;
or
Q¹ and Q² or Q² and Q³ together form a C₅₋₆heteroaryl or C₅₋₆heterocylclic ring; optionally containing one or two heteroatoms selected from N and O optionally substituted by any group selected from R⁵;
Z is selected from linear C₂₋₃ alkylene;
X³ is O;
X⁴ is selected from aryl, a 9-10 membered heteroaryl ring or a 9-10 membered heterocyclic ring, wherein the heteroaryl and heterocyclic rings contain one or more heteroatoms selected from N, and optionally additionally O,
   and wherein X⁴ is optionally substituted by one or two oxo moieties and is optionally substituted by one or more groups selected from R⁷;
with the proviso that
(i) when X⁴ is phenyl then Q¹ and Q² or Q² and Q³—together form an optionally substituted heteroaryl or heterocylclic ring as defined above; and
(ii) when Q¹, Q² and Q³ are independently selected from H or any group defined for R¹ and R² then X⁴ is not phenyl except when R² is C₁₋₅alkyl substituted by R³ wherein R³ is C₃₋₆heterocyclyl as defined above,
   their preparation and novel intermediates, compositions thereof and their use in the prevention or treatment of cardiac and cardiovascular disease and methods for the treatment thereof.

This invention relates to novel compounds and their preparation and use in treating cardiac and cardiovascular disease.

### BACKGROUND

β-adrenoceptor antagonists (β-blockers) are one of the most important therapies in the management of symptoms of, and for prolonging life in, cardiovascular disorders e.g. ischaemic heart disease and cardiac arrhythmias. They work by blocking the β1-adrenoceptors in the heart and thus prevent the endogenous hormones adrenaline and noradrenaline from increasing heart rate and force of contraction. β-blockers are also widely used in the management of hypertension, and (although the mechanism of action is not yet understood) they prolong life in patients with heart failure.

However, they are contraindicated in patients with respiratory disease (especially asthma and chronic obstructive pulmonary disease, COPD) because antagonism of the β2-adrenoceptors in the airways, results in bronchoconstriction and a loss of action of the important β2-agonist bronchodilators. Thus, currently many people (about 0.6% of the total adult population in the UK) with cardiovascular disease are unable to take β-blockers that would prolong their life and improve their cardiovascular symptoms, because of their concomitant respiratory disease. This is because the best β1-selective β-antagonist currently available for clinical use binds to the human β1-adrenoceptor with only 14 fold higher affinity than the human β2-adrenoceptor (Baker, 2005; Br. J Pharmacol: 144, 317-22).

Accordingly there is a need for beta blockers which are selective for just heart disease, ie have a high β₁/β₂ selectivity. Classes of phenoxypropanolamine compounds are known which are extended beyond the amine group and are substituted in the phenol ring. One particular class of phenoxypropanolamine compounds comprises a substituted ethylene dioxy substituent para to the phenyl moiety. This class which has never entered into clinical use includes the development compound LK-204545 with an phenyl(alkylurea) substituent to the amine moiety and with 1,778-fold β₁-selectivity:

and D-140S with a phenyl alkyl substituent to the amine moiety and with 4,400-fold β₁-selectivity:

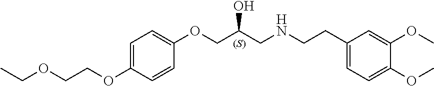

WO2008083054 discloses beta-1 adrenoreceptor selective ligands that find use as imaging agents within nuclear medicine applications. Compounds include an imaging moiety such as a radioactive moiety. The broadly disclosed class of compounds includes compounds having the core 1-phenoxy, 2-hydroxy propan-3-amine with extensive substitution of the phenoxy and amine moieties.

### BRIEF SUMMARY OF THE DISCLOSURE

We have now applied a multidisciplinary approach to beta receptor agonist and antagonist design to provide novel compounds which have significant selectivity for β₁-adrenoceptors and which have potential for clinical use.

### DETAILED DESCRIPTION

In accordance with the present invention there is provided a compound of Formula (I), and its pharmaceutically acceptable salt or salts and physiologically hydrolysable derivatives in free form or salt form:

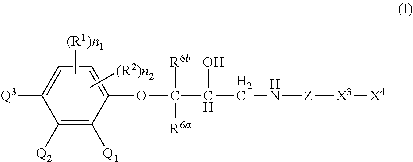

wherein
R¹ is independently selected from F, Cl, Br, CN, NH₂, OH, CHO, COOH, oxo, C₁₋₄alkoxy, CONH₂ (optionally mono- or di-substituted by C₁₋₄alkyl) and SO₂NH₂,
R² is independently selected from C₁₋₆allkyl substituted by R³ wherein the C₁₋₆alkyl chain optionally comprises one or two heteroatoms select from O;
R³ is selected from aryl, C₃₋₆cycloalkyl, C₃₋₆heterocyclyl and C₃₋₆heteroaryl, wherein the heterocyclyl and heteroaryl rings are nitrogen containing; and wherein R³ is optonally substituted by one or more groups selected from R¹;
n1 is zero or an integer from 1 to 2;
n2 is zero or an integer from 1 to 2;
and the sum of n1 and 2 is less than or equal to 2;
and wherein the

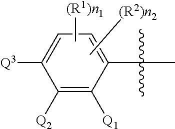

group contains at least one R² group either as a component of (R²)ₙ₂ or R⁵;
R⁵ is selected from any group defined for R¹ and R²;
R^{6a} and R^{6b} are independently selected from H or C₁₋₄alkyl;
R⁷ is independently selected from F, Cl, Br, CN, NH₂, OH, CHO, COOH, oxo, C₁₋₄alkyl, C₁₋₄alkoxy, CONK, (optionally mono- or di-substituted by C₁₋₄alkyl) and SO₂NH₂,
Q¹, Q² and Q³ are independently selected from H or any group defined for R¹ and R²; or
Q¹ and Q² or Q² and Q³ together form a C₅₋₆heteroaryl or C₅₋₆heterocylclic ring; optionally containing one or two heteroatoms selected from N and O optionally substituted by up to two groups selected from R⁵;
Z is selected from linear C₂₋₃ alkylene;
X³ is O;
X⁴ is selected from aryl, a 9-10 membered heteroaryl ring or a 9-10 membered heterocyclic ring, wherein the heteroaryl and heterocyclic rings contain one or more heteroatoms selected from N, and optionally additionally 0,
   and wherein X⁴ is optionally substituted by one or two oxo moieties and is optionally substituted by one or more groups selected from R⁷;
      with the proviso that:
(i) when X⁴ is phenyl then Q¹ and Q² or Q² and Q³—together form an optionally substituted heteroaryl or heterocylclic ring as defined above; and
(ii) when Q¹, Q² and Q³ are independently selected from H or any group defined for R¹ and R² then X⁴ is not phenyl except when R² is C₁₋₅alkyl substituted by R³ wherein R³ is C₃₋₆heterocyclyl as defined above,

In a further aspect of the invention there is provided a compound of Formula (I) as defined above;

with the proviso that:
(i) when X⁴ is phenyl then Q¹ and Q² or Q² and Q³—together form an optionally substituted heteroaryl or heterocylclic ring as defined above; and
(ii) when Q¹, Q² and Q³ are independently selected from H or any group defined for R¹ and R² then X⁴ is not phenyl.

In a further embodiment of the invention there is provided a compound of Formula (Ia) or Formula (Ib), and its pharmaceutically acceptable salt or salts and physiologically hydrolysable derivatives in free form or salt form:

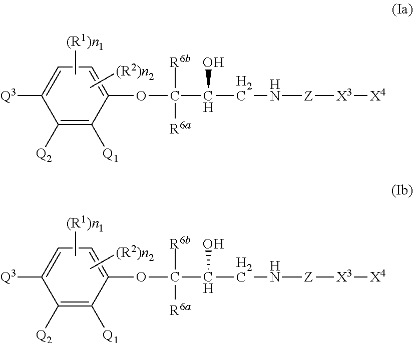

wherein R¹, R², n1, n2, Q¹, Q², Q³, R^{6a}, R^{6b}, Z, X³ and X⁴ are as defined above.

In accordance with a further aspect of the present invention there is provided a compound of Formula (II), and its pharmaceutically acceptable salt or salts and physiologically hydrolysable derivatives in free form or salt form:

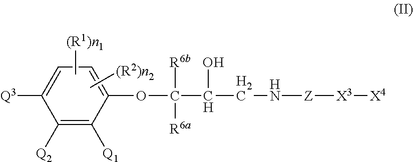

wherein
R¹ is independently selected from F, Cl, Br, CN, NH₂, OH, CHO, COOH, CONH₂ and SO₂NH₂,
R² is independently selected from NHR³, NO₂, CF₃, OR³, COR³, OCOR³, COOR³, COONR³₂, NR³COR³, CONR³₂, SO₂NR³₂, NR³SO₂R³, C₁₋₅alkyl, C₁₋₅alkoxy, C₂₋₅alkenyl, C₂₋₅alkynyl, —W—C₃₋₁₀cycloalkyl and —W—C₅₋₁₀carbocyclyl wherein W is C₁₋₅alkylene, C₂₋₅alkenylene or C₂₋₅alkynylene;
   any of which may comprise one or more carbonyl units or heteroatoms selected from O, S and N and which may be unsubstituted or further substituted by one of more R²¹;
R²¹ is independently selected from C₁₋₅alkyl, C₂₋₅alkenyl, C₂₋₅alkynyl and a group as defined for R¹
   wherein the C₁₋₅alkyl, C₂₋₅alkenyl, C₂₋₅alkynyl groups are optionally substituted by one or more groups independently selected from R¹;
R³ is independently selected C₁₋₅alkyl, C₁₋₅alkoxy, C₂₋₅alkenyl, C₂₋₅alkynyl, aryl, C₃₋₁₀cycloalkyl, and C₅₋₁₀-carbocyclyl;
   any of which may comprise one or more carbonyl units or heteroatoms selected from O, S and N and which may be unsubstituted or further substituted by one of more groups independently selected from R¹;
n1 and n2 and the sum thereof are independently selected from zero and a whole number integer from 1 to 2;
R⁵ is selected from a group as defined for R²;
R^{6a} and R^{6b} are independently selected from H or C₁₋₄alkyl or comprise part of a ring as defined below;
R⁷ is independently selected from F, Cl, Br, CN, NH₂, OH, CHO, COOH, CONH₂ and SO₂NH₂,
R⁸ is independently selected from NHR⁹, NO₂, CF₃, OR⁹, COR⁹, OCOR⁹, COOR⁹, COONR⁹₂, NR⁹COR⁹, CONR⁹₂, SO₂NR⁹₂, NR⁹SO₂R⁹, C₁₋₅alkyl, C₁₋₅alkoxy, C₂₋₅alkenyl, C₂₋₅alkynyl, —W—C₃₋₁₀cycloalkyl and —W—C₅₋₁₀-carbocyclyl wherein W is C₁₋₅alkylene, C₂₋₅alkenylene or C₂₋₅alkynylene;
   any of which may comprise one or more carbonyl units or heteroatoms selected from O, S and N and which may be unsubstituted or further substituted by one of more R⁸¹;
R⁸¹ is independently selected from C₁₋₅alkyl, C₂₋₅alkenyl, C₂₋₅alkynyl and a group as defined for R⁷
   wherein the C₁₋₅alkyl, C₂₋₅alkenyl, C₂₋₅alkynyl groups are optionally substituted by one or more groups independently selected from R⁷;
R⁹ is independently selected C₁₋₅alkyl, C₁₋₅alkoxy, C₂₋₅alkenyl, C₂₋₅alkynyl, aryl, C₃₋₁₀cycloalkyl, and C₅₋₁₀carbocyclyl;
   any of which may comprise one or more carbonyl units or heteroatoms selected from O, S and N and which may be unsubstituted or further substituted by one of more groups independently selected from R⁷;
Q¹, Q² and Q³ are independently selected from H or any group defined for R¹ and R²;
   or
Q¹, CR^{6a} and optionally R^{6b} together form an oxygen-containing heteroaryl or heterocylclic ring; optionally substituted by oxo and Q² and Q³ are independently selected from H, R¹ and R²;
   or
Q¹ is independently selected from H, R¹ and R² and Q² and Q³ together form an oxygen-containing heteroaryl or heterocylic ring optionally substituted by oxo and one, two of three groups selected from R⁵;
Z is selected from linear C₂₋₃ alkylene;
X³ is selected from O and S;
X⁴ is selected from aryl, a 5-15 membered heteroaryl ring or a 5-15 membered heterocyclic ring, wherein the heteroaryl and heterocyclic rings contain one or more heteroatoms selected from N, and optionally additionally O and for S,
   and wherein X⁴ is optionally substituted by one or two oxo moieties and is optionally substituted by one or more R⁷ and/or R⁸;
      with the proviso that:
(i) when n1 and n2 are both 0, or n1 is 1 or 2 and R¹ is cyano, methyl and/or chloro or n=2 and R² is allyloxy, Q¹ and Q² are both hydrogen, Q³ is 4-(2-methoxyethoxymethy), 4-(2-cyclopropylmethoxyethyl), 4-(2-isopropoxyethoxymethyl), 4-(2-n-butyloxyethoxymethyl), 4-(2-isobutoxyethyl), 4-(2-cyclobutylmethoxyethyl or 4-(2-phenoxyethoxymethyl), Z is ethylene or propylene, X³ is —O— then X⁴ cannot be unsubstituted pyridazin-3-one, unsubstituted 4,5-dihydropyndazin-3-one, pyridazin-3-one substituted by methyl or 4,5-dihydropyridazin-3-one substituted by methyl.
(ii) When n1=0, n2 is 0, Q¹ and Q² are both hydrogen, Q³ is R², R^{6a} and R^{6b} are both hydrogen, Z is ethylene, X³ is —O— and X⁴ is phenyl optionally substituted by methyl or methoxy then R² cannot be 1H-thieno[3,2-c)-pyrazolyl;
(iii) When R^{6a} and R^{6b} are both hydrogen, Z is ethylene, X³ is —O—, X⁴ is phenyl substituted by carbamoyl or acetylamino
   then

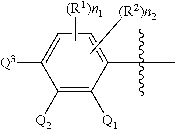

cannot form dihydrobenzofuranyl optionally substituted by methy or dimethyl; and cannot be phenyl substituted by 1-methyl-4-trifluoromethylimidazol-2-yl;
(iv) When R^{6a} and R^{6b} are both hydrogen, Z is ethylene, X³ is —O—, X⁴ is unsubstituted phenyl, 4-methoxyphenyl and 3,4-dimethoxyphenyl and Q³ is methoxyethoxy, cyclopropymethoxyethoxy and 4-methoxybenzyloxyethoxy then n1 cannot beg and when n1 is 1, R¹ cannot be 2-bromo or 2-cyano;
(v) The following compound is excluded: (S)-3-(2-cyanophenoxy)-N-(2-(2-fluorophenoxy)ethyl)-2-hydroxypropanol-1-aminium.

In another embodiment of the invention there is provided a compound of Formula (II) as defined above, with the proviso that
(i) X⁴ cannot be unsubstituted pyridazin-3-one or unsubstituted 4,5-dihydropyridazin-3-one pyridazin-3-one substituted by methyl or 4,5-dihydropyridazin-3-one substituted by methyl.
(ii) R² cannot be 1H-thieno[3,2-c)-pyrazolyl;

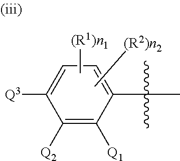

cannot form dihydrobenzofuranyl optionally substituted by methy or dimethyl; and cannot be phenyl substituted by 1-methyl-4-trifluoromethylimidazol-2-yl; and
(iv) When R^{6a} and R^{6b} are both hydrogen, Z ethylene, X³ is —O—, X⁴ is unsubstituted phenyl, 4-methoxyphenyl and 3,4-dimethoxyphenyl and Q³ is methoxyethoxy, cyclopropymethoxyethoxy and 4-methoxybenzyloxyethoxy then n1 cannot be 0 and when n1 is 1. R¹ cannot be 2-bromo or 2-cyano;
(v) The following compound is excluded: (S)-3-(2-cyanophenoxy)-N-(2-(2-fluorophenoxy)ethyl)-2-hydroxypropanol-1-aminium.

In a further embodiment of the invention there is provided a compound of Formula (IIa), and its pharmaceutically acceptable salt or salts and physiologically hydrolysable derivatives in free form or salt form:

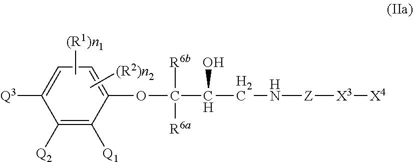

wherein R¹, R², n1, n2, Q¹, Q², Q³, R^{6a}, R^{6b}, Z, X³ and X⁴ are as defined above

In a further embodiment of the invention there is provided a compound of Formula (IIb), and its pharmaceutically acceptable salt or salts and physiologically hydrolysable derivatives in free form or salt form:

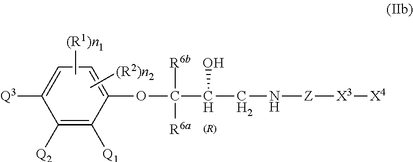

wherein R¹, R², n1, n2, Q¹, Q², Q³, R^{6a}, R^{6b}, Z, X³ and X⁴ are as defined above.

Abbreviations have the following meanings:

c. is cyclo, for example c.pr relates to cyclopropyl; i. is iso; Me is methyl; Pr or pr. is propyl; Bu or bu. is butyl; i-bu. is isobutyl; pent is pentyl; halo is F, Cl, Br or I; Ph is phenyl and Bz is benzyl; o, m and p are ortho, meta and para; subst. is substituted; o.s. is optionally substituted; - is unsubstituted;

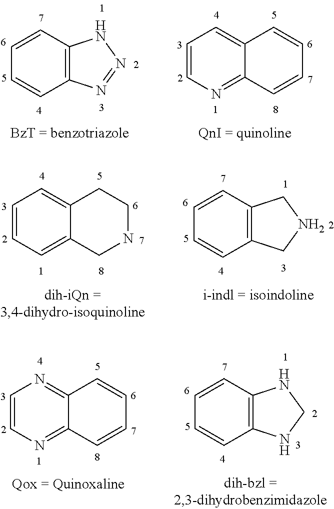 [SMILES: c1ccc2[nH]nnc2c1 ; c1ccc2ncccc2c1 ; c1ccc2c(c1)CCNC2 ; c1ccc2c(c1)CNC2 ; c1ccc2nccnc2c1 ; c1ccc2c(c1)NCN2]

It is to be understood that, insofar as certain of the compounds of Formula (I) or Formula (II) defined above and subformulae thereof may exist in optically active or racemic forms by virtue of one or more asymmetric carbon atoms, the invention includes in its definition any such optically active or racemic form which possesses adrenoceptor activity. The synthesis of optically active forms may be carried out by standard techniques of organic chemistry well known in the art, for example by synthesis from optically active starting materials or by resolution of a racemic form. Similarly, the above-mentioned activity may be evaluated using the standard laboratory techniques referred to hereinafter.

Examples of suitable methods for separating the enantiomers of a racemic compound include chromatography using a suitable chiral stationary phase; or conversion of a racemic mixture into diastereomeric derivatives, separation of the mixture of diastereomeric derivatives into two single diastereomers, and regeneration of a separate single enantiomer from each separate single diastereomer.

Examples of suitable methods for separating a mixture of diastereomers include fractional crystallisation, normal-phase chromatography, or reverse-phase chromatography.

It is to be understood that certain compounds of Formula (I) or Formula (II) defined above and subformulae thereof may exhibit the phenomenon of tautomerism. In particular, tautomerism may affect any heterocyclic groups that bear 1 or 2 oxo substituents. It is to be understood that the present invention includes in its definition any such tautomeric form, or a mixture thereof, which possesses β₁ adrenoceptor activity and is not to be limited merely to any one tautomeric form utilised within the formulae drawings or named in the Examples.

It is to be understood that certain compounds of Formula (I) or Formula (II) defined above and subformulae thereof may exist in unsolvated forms as well as solvated forms, such as, for example, hydrated forms. It is to be understood that the present invention encompasses all such solvated forms that possess β₁ adrenoceptor activity.

It is also to be understood that certain compounds of the Formula (I) or Formula (II) defined above and subformulae thereof may exhibit polymorphism, and that the present invention encompasses all such forms which possess β₁ adrenoceptor activity.

In this specification the generic term ‘alkyl’, unless specifically specified otherwise, includes both straight-chain and branched-chain alkyl groups such as propyl, isopropyl and tert-butyl. However, references to individual alkyl groups such as ‘propyl’ are specific for the straight-chain version only, and references to individual branched-chain alkyl groups such as ‘isopropyl’ are specific for the branched-chain version only. The same principle also applies to generic terms ‘alkenyl’ and ‘alkynyl’, and other alkyl containing groups such as alkoxy, unless specified otherwise.

The term ‘aryl’ refers to phenyl or naphthyl.

The term ‘cycloalkyl’ refers to a 3-12 membered mono, bi or tricyclic saturated carbon ring, for example a mono or bicyclic saturated carbon ring. Examples of cycloalkyl include cyclopropy and cyclopentyl.

The term ‘carbocyclyl’ refers to a 5-12 membered, preferably 5-10 membered unsaturated or partially unsaturated carbon ring. Examples of carbocyclyl include: phenyl, naphthyl and indene. For the avoidance of doubt when a carbocycl ring optionally comprises one or more heteroatoms as in the definition of R₂, then the heteroatom(s) replace carbon atoms such that C₁₀carbocycl comprising a nitrogen atom includes quinolinyl.

The term ‘heterocyclyl’ or ‘heterocyclic ring’ refers to a 5-12 membered, preferably 5-10 membered saturated or partially saturated mono or bicyclic ring, said saturated or partially unsaturated rings containing up to 5 heteroatoms independently selected from nitrogen, oxygen or sulphur, linked via ring carbon atoms or ring nitrogen atoms where a bond from a nitrogen is allowed. This definition further comprises sulphur-containing rings wherein the sulphur atom has been oxidised to an S(O) or S(O2) group. Examples of saturated or partially saturated heterocyclic rings include isoindolinyl, chromanyl, tetrahydroisoquinolinyl, benzodioxanyl and benzimidazolinyl.

The term ‘heteroaromatic ring’ or ‘heteroaryl’ refers to a 5-10 membered, for example, 9-10 membered and 5-6 membered, aromatic ring containing from 1 to 4 heteroatoms independently selected from O, N and S. Example of such ‘heteroaromatic’ rings include: quinolinyl, benzimidazolyl and quinazolinyl.

Preferred compounds of Formula (I) or Formula (II) and its sub-formulae are those wherein any one of the following or any combination of the following applies:
(i) R¹ is chloro, bromo or fluoro, C₁₋₄alkyl (optionally substituted by amino, C₁₋₄alkoxy, C₁₋₄ perfluoroalkyl or cyano;
(ii) R¹ is chloro, bromo or fluoro, C₁₋₄alkyl, C₁₋₄alkoxy, CF₃, C₂F₅ or cyano;
(iii) R¹ is methyl, methoxy, trifluoromethyl, fluoro, aminomethyl or cyano,
(iv) R¹ is chloro, bromo or fluoro, C₁₋₄alkyl, C₁₋₄alkoxy or cyano;
(v) R¹ is chloro, bromo, C₁₋₄alkyl, or cyano;
(vi) R¹ is chloro or bromo;
(vii) R¹ is fluoro;
(viii) R¹ is cyano;
(ix) R¹ is hydroxyl, fluoro, chloro, carboxy, —CONH₂, CONH(C₂H₅), —CON(CH₃)₂, —NHCOCH₃ or —SO₂CH₃;
(x) R² is independently selected from C₁₋₅alkyl, C₁₋₅alkoxyC₀₋₅alkyl(O)₀₋₁, C₂₋₅alkenyl, C₂₋₅alkynyl, aryl, C₃₋₈cycloalkyl, C₅₋₁₀carbocyclyl, C₅₋₁₀heterocyclyl and C₅₋₁₀heteroaryl; or
   C₁₋₅alkyl, C₀₋₅alkoxyC₀₋₅alkyl(O)₀₋₁, C₂₋₅alkenyl or C₂₋₅alkynyl substituted by aryl, C₃₋₈cycloalkyl, C₅₋₁₀carbocyclyl, C₅₋₁₀heterocyclyl or C₅₋₁₀heteroaryl;
   said chain or ring may be unsubstituted or substituted by one or more oxo or R²¹;
(xi) R² is aryl which may be unsubstituted or further substituted by one of more R²¹;
(xii) R² is C₀₋₅alkoxyC₀₋₅alkyl substituted by aryl or C₃₋₆cycloalkyl;
(xiii) R² is C₁₋₃alkoxyC₃₋₅alkyl substituted by aryl or C₃₋₆cycloalkyl;
(xiv) R² is C₁₋₅alkoxy substituted by aryl, for example phenyl;
(xv) R² is C₁₋₆alkyl, for example 3-methylbutyl;
(xvi) R² is C₃₋₆cycloalkylC₁₋₅alkyl or phenylC₁₋₅alkyl where the C₁₋₅alkyl optionally contains 1 or 2 heteroatoms selected from O;
(xvii) R² is C₃₋₆cycloalkylC₁₋₅alkyl where the C₁₋₅alkyl optionally contains 1 or 2 heteroatoms selected from O, for example C₁₋₅alkoxy optionally further comprising one additional heteroatom selected from O;
(xviii) R² is phenylC₁₋₅alkyl where the C₁₋₅alkyl optionally contains 1 or 2 heteroatoms selected from O, for example C₁₋₅alkoxy optionally further comprising one additional heteroatom selected from O;
(xix) R² is C₃₋₆heterocyclylC₁₋₅alkyl or C₃₋₆heteroarylC₁₋₅alkyl where the C₁₋₅alkyl optionally contains 1 or 2 heteroatoms selected from O, for example C₁₋₅alkoxy optionally further comprising one additional heteroatom selected from O;
(xx) R² is cyclopropylmethoxymethyl, cyclopropylmethoxyethyl, methyl, 3-methylbutyl, methoxy, cyclopropylmethoxymethyl, cyclopropylmethoxypropyl, cyclopentoxymethyl, cyclopentoxyethoxy, cyclopentoxypropyl, phenylethoxyethoxy optionally substituted by fluoro and phenylethoxy optionally substituted by fluoro;
(xxi) R² is methyl, 3-methylbutyl, methoxy, cyclopropylmethoxymethyl, cyclopropylmethoxyethyl, cyclopropylmethoxypropyl, cyclopentoxymethyl, cyclopentoxyethoxy, cyclopentoxypropyl, phenylethoxyethoxy optionally substituted by fluoro and phenylethoxy optionally substituted by fluoro;
(xxii) R² is trifluoromethyl, trifluoromethoxy, methoxy, phenyl, benzyl or benzyloxy;
(xxiii) R² is cyclopentyloxyethoxy, cyclopropylmethoxypropyl, cyclopentoxypropyl, cyclopropylmethoxyethyl, cyclopropylmethoxy, cyclopropylmethoxymethyl and cyclopropoxyethyl.
(xxiv) For the avoidance of doubt C₁₋₅alkoxyC₀₋₅alkyl(O)₀₋₁ in R² cannot be C₁₋₅alkoxy-O—;
(xxv) R⁵ is a group selected from R².
(xxvi) R⁵ or R² is selected from phenoxyC₁₋₄alkyl optionally substituted by upto four groups selected from C₁₋₄alkyl, cyano, halo (such as fluoro), C₁₋₄alkoxy (optionally substituted by upto 5 fluoro groups).
(xxvii) R⁵ or R² is selected from phenoxyC₁₋₂alkyl optionally substituted by upto four groups selected from C₁₋₂alkyl, cyano, halo (such as fluoro), C₁₋₂alkoxy (optionally substituted by upto 5 fluoro groups),
(xxviii) n1 is 1;
(xxix) n1 is 0
(xxx) n2 is 1;
(xxxi) n2 is 0;
(xxxii) Q¹ and Q² or Q² and Q³ together form a C₅₋₆heteroaryl or C₅₋₆heterocylclic ring; optionally containing one or two heteroatoms selected from N and O optionally substituted by up to two groups selected from R⁵;
(xxxiii) Q¹ is hydrogen;
(xxxiv) Q² is hydrogen;
(xxxv) Q³ is C₃₋₆cycloalkyl-(CH₂)₀₋₂—O—(CH₂)₀₋₄—;

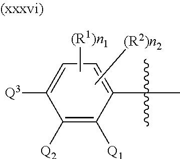

forms the group

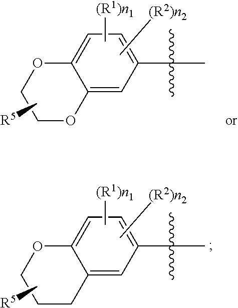

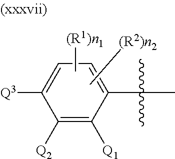

forms the group

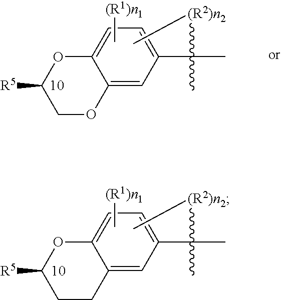

(xxxviii) R^{6a} is hydrogen;
(xxxix) R^{6b} is hydrogen;
(xl) Z is ethylene;
(xli) Z is propylene;
(xiii) X³ is —O—;
(xliii) X³ is —S—;
(xliv) X⁴ is phenyl;
(xiv) X⁴ is a 9-10 membered heteroaryl ring;
(xlvi) X⁴ is a 9-10 membered heteroaryl ring selected from benztriazole, quinoline and quinoxaline;
(xlvii) X⁴ is a 9-10 membered heterocyclic ring;
(xlviii) X⁴ is a 9-10 membered heterocyclic ring selected from indole, isoindole, indoline, isoindoline, indolizine, indazole, 3,4-dihydroisoquinoline, cinnoline, quinoline, quinoxaline, phthalazine, quinazoline, naphthyridine, benzthiazole, benzotriazole, benzthiazole, benzimidazole and 2,3-dihydroxybenzimidazole.
(xlix) X⁴ is a 9-10 membered heterocyclic ring selected from 3,4-dihydroisoquinoline, quinoline, quinoxaline, benzotriazole, isoindoline and 2,3-dihydroxybenzimidazole,
(l) X⁴ is a 9-10 membered heterocyclic ring selected from isoindoline and 2,3-dihydroxybenzimidazole;
(li) R² or is the group 4-R⁴(O)ₙ₃Z¹(O)ₙ₄ wherein:
   R⁴ is selected from unsubstituted and substituted C₁-C₈ linear or branched alkyl, C₂₋₅ alkenyl, C₆-C₁₀ heteroaryl or aryl, C₃-C₈ cycloalkyl or heterocyclyl which may be part unsaturated, and combinations thereof, wherein substituents are as hereinbefore defined for R¹ and R²;
   n3 and n4 are independently selected from 0 and the whole number integer and
   Z¹ is C₁-C₄ branched or linear alkyl or alkenyl,
(lii) R⁴ is selected from unsubstituted and substituted C₃₋₇ cycloalkyl C₀₋₃ alkyl, and C₃₋₇ cycloalkyl;
(liii) R⁴ is selected from c.prCH₂ and cyclopentyl;
(liv) n3 is 1 and n4 is
(lv) 4-R⁴OZ¹O if present, is selected from 4-cyclopropylmethoxypropoxy and 4-cyclopropylmethoxy;
(lvi) R² is independently selected from C₁₋₅alkyl, C₁₋₅alkoxyC₀₋₅alkyl(O)₀₋₁, C₂₋₅alkenyl, C₂₋₅alkynyl, aryl, C₃₋₈cycloalkyl, C₅₋₁₀carbocyclyl, C₅₋₁₀heterocyclyl and C₅₋₁₀heteroaryl; or
   C₁₋₅alkyl, C₀₋₅alkoxyC₀₋₅alkyl(O)₀₋₁, C₂₋₅alkenyl or C₂₋₅alkynyl substituted by aryl, C₃₋₈cycloalkyl, C₅₋₁₀carbocyclyl, C₅₋₁₀heterocyclyl or C₅₋₁₀heteroaryl;
   said chain or ring may be unsubstituted or substituted by one or more oxo or R²¹;
(lvii) R⁷ and R⁸ are selected from NH₂, CF, R⁹ and NHCOOR⁹;
(lviii) R⁷ and R⁸ are selected from NH₂, CF₃, R⁹ and NHCOOR⁹ wherein R⁹ is CH₃;
(lix) R⁷ is selected from amino, carboxy, halo, C₁₋₄alkyl, C₁₋₄alkoxy, C₁₋₂perfluoroalkyl, oxo, —NHC(O)C₁₋₄alkyl or —CONH₂
(lx) R⁷ is selected from oxo or —CONH₂

In one preferred selection embodiment there is provided a compound of Formula (IIc) and its pharmaceutically acceptable salt or salts and physiologically hydrolysable derivatives:

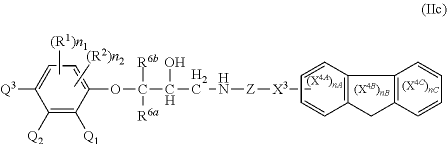

wherein R¹, R², n1, n2, Q¹, Q², Q³, R^{6a}, R^{6b}, Z and X³ are as defined above and nA, nB and nC are each selected from 0 and 1 and the sum thereof totals 1, 2 or 3 and:
nA, nB=1, nC=0; or
nA, nB, nC=1; or
nA, nC=1, nB=0; or
nB=1, nA, nC=0; and
X^{4A}, X^{4B} and X^{4C} are unsaturated rings wherein;
X^{4C} comprises one or more heteroatoms selected from N, O and S, and combinations thereof, and optionally additionally one or two oxo moieties; optionally comprises one or more heteroatoms selected from N; and
X^{4A}, X^{4B} and X^{4C} are optionally independently substituted by R⁷ and R⁸ wherein R⁷ and R⁸ are as defined above:

In one preferred selection embodiment there is provided a compound of Formula (IId) and its pharmaceutically acceptable salt or salts and physiologically hydrolysable derivatives:

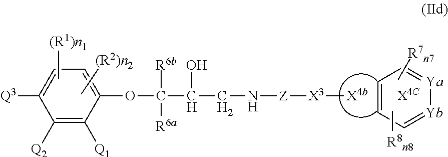

wherein R¹, R², n1, n2, Q¹, Q², Q³, R^{6a}, R^{6b}, R⁷, Z and X³ are as defined above and
X^{4B} is selected from 5 and 7 membered heterocyclic and heteroaromatic rings comprising one or two or three N heteroatoms and optionally one carbonyl moiety;
Ya and Yb are independently selected from —N— and —CH—;
R⁸ is as defined for R⁷; and
n7 and n8 and the sum thereof are independently selected from zero and the whole number integer from 1 to 4.

A compound as hereinbefore defined may be in free form, i.e. normally as a base, or in any suitable salt or ester form. Free forms of the compound may be converted into salt or ester form and vice versa, in conventional manner. Suitable salts include hydrochloride, dihydrochloride, hydroformate, amide, succinate, half succinate, maleate, acetate, trifluoroacetate, fumarate, phthalate, tetraphthalate, benzoate, sulfonate, sulphate, phosphate, oxalate, malonate, hydrogen malonate, ascorbate, glycolate, lactate, malate, tartarate, citrate, aspartate or glutamate and variants thereof. Suitable acids for acid addition salt formation include the corresponding acids, i.e. hydrochloric, formic, amino acid, succinic, maleic, acetic, trifluoroacetic, fumaric, phthalic, tetraphthalic, benzoic, sulfonic, sulphuric, phosphoric, oxalic, malonic, ascorbic, glycolic, lactic, malic, tartaric, citric, aspartic or glutamic acids and the like.

Suitable esters include those obtained with the above acids, with hydroxides such as sodium, potassium, calcium or the like, or with alcohols.

The compounds of Formula (I) or Formula (II) as defined above and subformulae thereof are optically active and may be prepared as one or both enantiomeric or tautomeric forms, or stereo or geometric isomeric forms, where relevant. Such forms may be identified and prepared or isolated by methods known in the art. Reference herein to compounds of Formula (I) or Formula (II) as defined above also encompasses reference to crystalline forms, polymorphs, hydrous and anhydrous forms and prodrugs thereof.

A compound of Formula (I) or Formula (II) as defined above or subformulae as hereinbefore defined, can be prepared by a process comprising a step selected from (a) to (e) as follows, these processes are provided as a further feature of the invention:—

(a) Reaction of a compound of formula Pr1 with a compound of formula Pr2,

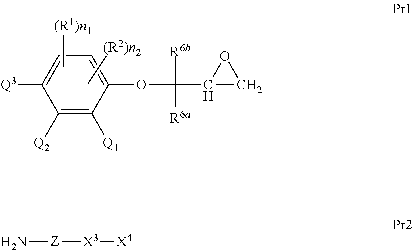

(b) Reaction of a compound of formula Pr3 with a compound of formula Pr4,

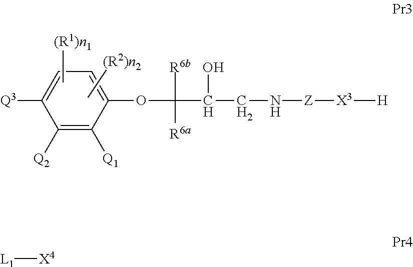

wherein L₁ is a leaving group;
   (c) Reaction of a compound of formula Pr5 with a compound of formula Pr6,

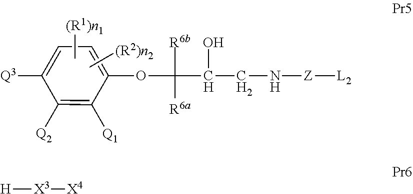

wherein L₂ is a leaving group;
   (d) Reaction of a compound of formula Pr7 with a compound of formula Pr8;

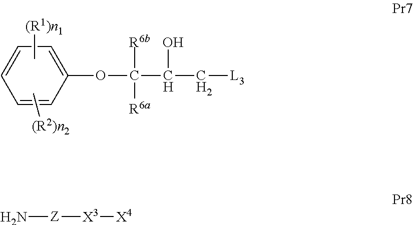

wherein L₃ is a leaving group
   (e) Reaction of a compound of formula Pr5 with a compound of formula Pr6

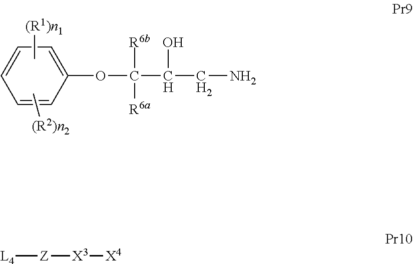

wherein L₄ is a leaving group
   and thereafter if necessary:
(i) converting a compound of Formula (I) or Formula (I into another compound Formula (I) or Formula (II);
(ii) removing any protecting groups; and/or
(iii) forming a salt, pro-drug or solvate.

Intermediates for the preparation of compounds of Formula (I) or Formula (II), as described above can be prepared as follows:

### Process a)

(i) A compound of formula Pr1 is conveniently prepared by methods described in International patent application number: WO 2012/004549 from the corresponding phenol of formula In1 or is commercially available:

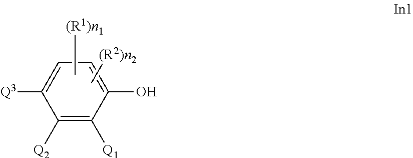

(ii) In1 is conveniently obtained by interchange from a commercially available analogue or is commercially available,
(iii) A compound of formula Pr2 is commercially available or prepared by processes well known to a person skilled in the
   Process b)-e)

Processes b) e) are conducted using methodologies well know to the skilled man and the intermediates used therein are either commercially available or made by methodologies well know to the skilled man

Suitably a process is as hereinbefore defined or as hereinbelow illustrated in the drawings.

In a further aspect of the invention there is provided a novel intermediate as hereinbefore defined.

In a further aspect of the invention there is provided a process as hereinbefore defined for the preparation of a novel intermediate as hereinbefore defined or as hereinbelow illustrated in the drawings.

### Therapeutic Use

In a further aspect of the invention there is provided a compound of Formula (I) or Formula (II) or subformulae as hereinbefore defined for use as a medicament.

In a further aspect of the invention there is provided the use of a compound of Formula (I) or Formula (II) or subformulae as hereinbefore defined in the prevention or treatment of a condition selected from ischaemic heart disease (also known as myocardial infarction or angina), hypertension and heart failure, restenosis and cardiomyopathy, more preferably with concomitant respiratory disease, in particular asthma or COPE).

In a further aspect of the invention there is provided the use of a compound of Formula (I) or Formula (II) or subformulae as hereinbefore defined in the manufacture of a medicament for prevention or treatment of a condition selected from ischaemic heart disease (also known as myocardial infarction or angina), hypertension and heart failure, restenosis and cardiomyopathy, more preferably with concomitant respiratory disease, in particular asthma or COPD.

In a further aspect of the invention there is provided a compound of Formula (I) or Formula (II) or subformulae as hereinbefore defined for the prevention or treatment of a condition selected from ischaemic heart disease (also known as myocardial infarction or angina), hypertension and heart failure, restenosis and cardiomyopathy, more preferably with concomitant respiratory disease, in particular asthma or COPD.

In a further aspect of the invention there is provided a method of treating a condition selected from ischaemic heart disease (also known as myocardial infarction or angina), hypertension and heart failure, restenosis and cardiomyopathy, more preferably with concomitant respiratory disease, in particular asthma or COPD, said method comprising administering to a subject in need thereof, a compound of Formula (I) or Formula (II) or subformulae or pharmaceutically acceptable salt thereof as hereinbefore defined in an amount sufficient to treat the condition.

In a further aspect of the invention there is provided a method of preventing a condition selected from ischaemic heart disease (also known as myocardial infarction or angina), hypertension and heart failure, restenosis and cardiomyopathy, more preferably with concomitant respiratory disease, in particular asthma or COPD, said method comprising administering to a subject in need thereof, a compound of Formula (I) or Formula (II) or subformulae or pharmaceutically acceptable salt thereof as hereinbefore defined in an amount sufficient to treat the condition.

The use of a compound of the invention in the manufacture of a medicament as hereinbefore defined includes the use of the compound directly, or in any stage of the manufacture of such a medicament, or in vitro in a screening programme to identify further agents for the prevention or treatment of the hereinbefore defined diseases or conditions.

A further aspect of the invention relates to the use of a compound of Formula (I) or Formula (II) or subformulae or a pharmaceutically acceptable salt or solvate or physiologically hydrolysable, solubilising or immobilising derivative thereof, in an assay for identifying candidate compounds capable of treating one or more disorders or diseases as hereinbefore defined.

### Pharmaceutical Compositions

In a further aspect of the invention there is provided a composition comprising a therapeutically effective amount of a compound of Formula (I) or Formula (II) or subformulae or its pharmaceutically acceptable salt or physiologically hydrolysable derivative as hereinbefore defined in association with one or more pharmaceutical carriers, excipients or diluents. Suitable carriers, excipients or diluents may be selected having regard to the intended mode of administration and standard practice. The pharmaceutical compositions may be for human or animal usage in human and veterinary medicine, preferably for treatment of a condition, disease or disorder as hereinbefore defined

Examples of suitable carriers include lactose, starch, glucose, methyl cellulose, magnesium stearate, mannitol, sorbitol and the like.

A composition or compound of the invention is suitably for any desired mode of administration including oral, rectal, vaginal, parenteral, intramuscular, intraperitoneal, intraarterial, intrathecal, intrabronchial, subcutaneous, intradermal, intravenous, nasal, buccal or sublingual and the like. An indicated daily dosage is from about 1 mg to about 500 mg and compositions for oral administration generally contain from about 0.25 mg to about 250 mg of the compound together with solid or liquid carriers and diluents. A therapeutically effective amount is any amount from 0.1% to 99.9% w/w.

A composition for oral administration is suitably formulated as a compressed tablet, tablet, capsule, gel capsule, powder, solution, dispersion, suspension or the like. Such forms may be produced according to known methods and may include any suitable binder, lubricant, suspending agent, coating agent or solubilising agent or combinations thereof.

A composition for administration by means of injection is suitably formulated as a sterile solution or emulsion from a suitable solution or powder. Alternatively a composition may be in the form of suppositories, pessaries, suspensions, emulsions, lotions, creams, ointments, skin patches, gels, solgels, sprays, solutions or dusting powders.

A composition may include one or more additional active ingredients or may be administered together with compositions comprising other active ingredients for the same or different condition. An additional active ingredient is suitably selected from a diuretic, calcium channel antagonist, angiotensin converting enzyme (ACE) inhibitor, angiotensin receptor antagonist and the like.

The compounds of the invention may be administered in the form of a pro-drug, that is a compound that is physiologically hydrolysable in the human or animal body to release a compound of the invention. A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in vivo cleavable ester derivatives that may be formed at a carboxy group or a hydroxy group in a compound of the Formula (I) or Formula (II) or subformulae as hereinbefore defined and in vivo cleavable amide derivatives that may be formed at a carboxy group or an amino group in a compound of the Formula (I) or Formula (II) or subformulae as hereinbefore defined.

Accordingly, the present invention includes those compounds of the Formula (I) or Formula (II) or subformulae as defined hereinbefore when made available by organic synthesis and when made available within the human or animal body by way of cleavage of a pro-drug thereof. Accordingly, the present invention includes those compounds of the Formula (I) or Formula (II) or subformulae as hereinbefore defined that are produced by organic synthetic means and also such compounds that are produced in the human or animal body by way of metabolism of a precursor compound, that is a compound of the Formula (I) or Formula (II) or subformulae as hereinbefore defined that may be a synthetically-produced compound or a metabolically-produced compound.

A suitable pharmaceutically-acceptable pro-drug of a compound of the Formula (I) or Formula (II) or subformulae as hereinbefore defined is one that is based on reasonable medical judgement as being suitable for administration to the human or animal body without undesirable pharmacological activities and without undue toxicity.

Various forms of pro-drug have been described, for example in the following documents:
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 Design and Application of Pro-drugs', by H, Bundgaard p, 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988);
f) N. Kakeya, et al., Chem. Pharm. Bull., 32, 692 (1984);
g) T. Higuchi and V. Stella, ‘Pro-Drugs as Novel Delivery Systems’, A.C.S. Symposium Series, Volume 14; and
h) E. Roche (editor). ‘Bioreversible Carriers in Drug Design’, Pergamon Press, 1987.

A suitable pharmaceutically-acceptable pro-drug of a compound of the Formula (I) or Formula (II) or subformulae as hereinbefore defined that possesses a carboxy group is, for example, an in vivo cleavable ester thereof. An in vivo cleavable ester of a compound of the Formula (I) or Formula (II) or subformulae as hereinbefore defined containing a carboxy group is, for example, a pharmaceutically-acceptable ester which is cleaved in the human or animal body to produce the parent acid. Suitable pharmaceutically-acceptable esters for carboxy include C₁₋₆alkyl esters such as methyl, ethyl and tert-butyl, C₁₋₆alkoxymethyl esters such as methoxymethyl esters. C₁₋₆alkanoyloxymethyl esters such as pivaloyloxymethyl esters, 3-phthalidyl esters, C₃₋₈cycloalkylcarbonyloxy-C₁₋₆alkyl esters such as cyclopentylcarbonyloxymethyl and 1-cyclohexylcarbonyloxyethyl esters, 2-oxo-1,3-dioxolenylmethyl esters such as 5-methyl-2-oxo-1,3-dioxolen-4-ylmethyl esters and C₁₋₆alkoxycarbonyloxy-C₁₋₆alkyl esters such as methoxycarbonyloxymethyl and 1-methoxycarbonyloxyethyl esters.

A suitable pharmaceutically-acceptable pro-drug of a compound of the Formula (I) or Formula (II) or subformulae as hereinbefore defined that possesses a hydroxy group is, for example, an in vivo cleavable ester or ether thereof.

An in vivo cleavable ester or ether of a compound of the Formula (I) or Formula (II) or subformulae as hereinbefore defined containing a hydroxy group is, for example, a pharmaceutically-acceptable ester or ether which is cleaved in the human or animal body to produce the parent hydroxy compound. Suitable pharmaceutically-acceptable ester forming groups for a hydroxy group include inorganic esters such as phosphate esters (including phosphoramidic cyclic esters). Further suitable pharmaceutically-acceptable ester forming groups for a hydroxy group include C₁₋₁₀alkanoyl groups such as acetyl, benzoyl, phenylacetyl and substituted benzoyl and phenylacetyl groups, C₁₋₁₀alkoxycarbonyl groups such as ethoxycarbonyl, N,N-[di-C₁₋₄alkyl]carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-C₁₋₄alkylpiperazin-1-ylmethyl, Suitable pharmaceutically-acceptable ether forming groups for a hydroxy group include alpha-acyloxyalkyl groups such as acetoxymethyl and pivaloyloxymethyl groups.

A suitable pharmaceutically-acceptable pro-drug of a compound of the Formula (I) or Formula (II) that possesses a carboxy group is, for example, an in vivo cleavable amide thereof, for example an amide formed with an amine such as ammonia, a C₁₋₄alkylamine such as methylamine, a di-C₁₋₄alkylamine such as dimethylamine. N-ethyl-N-methylamine or diethylamine, a C₁₋₄alkoxy-C₂₋₄alkylamine such as 2-methoxyethylamine, a phenyl-C₁₋₄alkylamine such as benzylamine and amino acids such as glycine or an ester thereof.

A suitable pharmaceutically-acceptable pro-drug of a compound of the Formula (I) or Formula (II) or subformulae as hereinbefore defined that possesses an amino group is, for example, an in vivo cleavable amide derivative thereof. Suitable pharmaceutically-acceptable amides from an amino group include, for example an amide formed with C₁₋₁₀alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁₋₄)alkylpiperazin-1-ylmethyl.

In a further aspect of the invention there is provided the use of a compound of Formula (I) or Formula (II) or subformulae or a composition as hereinbefore defined in the prevention or treatment of a cardiac or cardiovascular disease or condition preferably selected from ischaemic heart disease (also known as myocardial infarction or angina), hypertension and heart failure. In a particular advantage a compound or composition of the invention may be administered to a subject with, or used in the prevention or treatment of a subject suffering from one of the above diseases or conditions and from respiratory disease, in particular from asthma or COPD. In a further advantage a compound or composition of the invention may be administered to a subject with, or used in the prevention or treatment of a subject suffering from one of the above diseases or conditions and intolerant to a side effect associated with known beta blockers, in a further advantage a compound or composition of the invention has good oral bioavailability.

We have found that the compounds and compositions of the invention block beta-1 mediated responses but have substantially no affect on beta-2 mediated responses in a conscious animal. The beta-1 mediated responses include tachycardia, reflex heart rate response etc and the like, and are implicated in the above conditions. The beta-2 mediated responses include peripheral vascular conductance, hypotension and the like and are implicated in respiratory conditions.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

The invention will now be illustrated with the following non-limiting examples shown in Table 1:

TABLE 1
Description of compounds of formula II wherein n1 and n2 are both 0:

| | |
|---|---|
| | (II) |
| 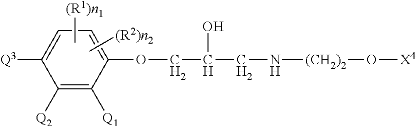 | |

| Ex | Q¹ | Q² | Q³ | 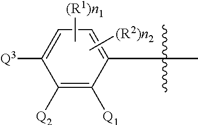 | R⁵ | X4 | R⁷, R⁸ | enanti- omer |
|---|---|---|---|---|---|---|---|---|
| 1 | H | H | 4-(2- | | | Ph | 4-CONH₂ | rac. |
| | | | (c. pentyloxy)eth- | | | | | |
| | | | oxy) | | | | | |
| 2 | H | H | 4-(2- | | | Ph | 4-F | rac. |
| | | | (c. pentyloxy)eth- | | | | | |
| | | | oxy) | | | | | |
| 3 | H | H | 4-(2- | | | Ph | 4-OMe | rac. |
| | | | (c. pentyloxy)eth- | | | | | |
| | | | oxy) | | | | | |
| 4 | H | H | 4-(2- | | | 6-dih- | 2-oxo | (S) |
| | | | (c. pentyloxy)eth- | | | iQn | | |
| | | | oxy) | | | | | |
| 5 | H | H | 4-(2- | | | Ph | 4-CONHMe | (S) |
| | | | (c. pentyloxy)eth- | | | | | |
| | | | oxy) | | | | | |
| 6 | H | H | 4-(2- | | | Ph | 4-CONMe₂ | (S) |
| | | | (c. pentyloxy)eth- | | | | | |
| | | | oxy) | | | | | |
| 7 | H | H | 4-(2- | | | Ph | 4-CONHEt | (S) |
| | | | (c. pentyloxy)eth- | | | | | |
| | | | oxy) | | | | | |
| 8 | H | H | 4- | — | — | Ph | 4-CONH₂ | (S) |
| | | | c. prCH₂O(CH₂)₃ | | | | | |
| 9 | H | H | 4- | — | — | Ph | 4-CONH₂ | (S) |
| | | | c. pentO(CH₂)₃ | | | | | |
| 10 | | | — | 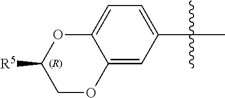 | c. prCH₂OCH₂ | Ph | 3-CONH₂ 4-OH | (R, S) |
| 11 | | | — | 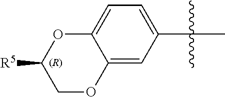 | c. prCH₂OCH₂ | Ph | 4-CONH₂ | (R, S) |
| 12 | H | H | 4- | — | — | 6-Qnl | — | (S) |
| | | | c. prCH₂O(CH₂)₃ | | | | | |
| 13 | H | H | 4- | — | — | Ph | 4- | (S) |
| | | | c. prCH₂O(CH₂)₃ | | | | NHCOCH₃ | |
| 14 | | | — | 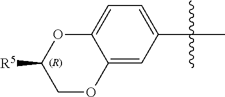 | c. prCH₂OCH₂ | 6-Qnl | — | (R, S) |
| 15 | | | — | 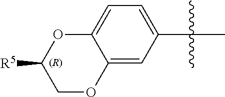 | c. prCH₂OCH₂ | Ph | 4- NHCOCH₃ | (R, S) |
| 16 | | | — | 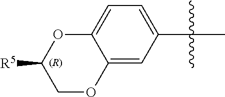 | c. pentOCH₂ | Ph | 4-CONH₂ | (R, S) |
| 17 | H | H | 4- | — | — | 5-i-indl | 1-oxo | (S) |
| | | | c. prCH₂O(CH₂)₃ | | | | | |
| 18 | | | | 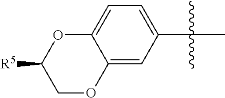 | c. prCH₂OCH₂ | 5-i-indl | 1-oxo | (R, S) |
| 19 | | | — | 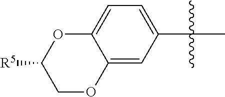 | c. prCH₂OCH₂ | Ph | 4-CONH₂ | (S, S) |
| 20 | H | H | 4- | — | — | Ph | 4-CONH₂ | (S) |
| | | | c. prCH₂O(CH₂)₂ | | | | | |
| 21 | H | H | 4- | — | — | 6-Qox | — | (S) |
| | | | c. prCH₂O(CH₂)₃ | | | | | |
| 22 | H | H | 4- | — | — | 5-dih- | 2-oxo | (S) |
| | | | c. prCH₂O(CH₂)₃ | | | bzl | | |
| 23 | | | — | 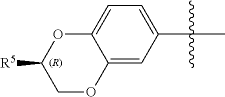 | c. prCH₂OCH₂ | 6-Qox | — | (R, S) |
| 24 | | | — | 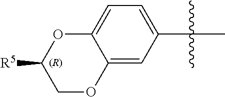 | c. prCH₂OCH₂ | 5-BzT | — | (R, S) |
| 25 | | | | 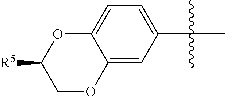 | 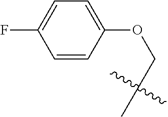 [SMILES: *COc1ccc(F)cc1] | 5-i-indl | 1-oxo | (R, S) |
| 26 | | | | 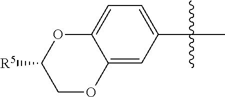 | 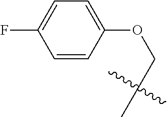 [SMILES: *COc1ccc(F)cc1] | 5-i-indl | 1-oxo | (S, S) |
| 27 | | | c. prCH₂O | 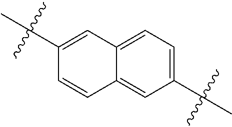 [SMILES: *c1ccc2cc(*)ccc2c1] | | Ph | 4-CONH₂ | (S) |
| 28 | | | c. prCH₂OCH₂ | 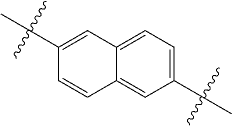 [SMILES: *c1ccc2cc(*)ccc2c1] | | Ph | 4-CONH₂ | (S) |
| 29 | | | c. prCH₂O | 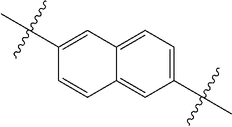 [SMILES: *c1ccc2cc(*)ccc2c1] | | 5-i-indl | 1-oxo | (S) |
| 30 | | | | 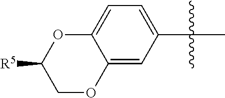 | 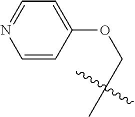 [SMILES: *COc1ccncc1] | 5-i-indl | 1-oxo | (R, S) |
| 31 | | | | 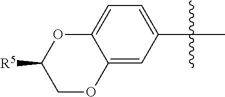 | 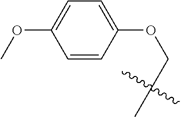 [SMILES: *COc1ccc(OC)cc1] | 5-i-indl | 1-oxo | (R, S) |
| 32 | | | | 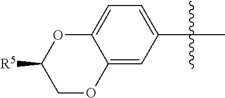 | 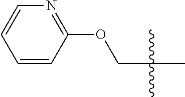 [SMILES: *COc1ccccn1] | 5-i-indl | 1-oxo | (R, S) |
| 33 | | | | 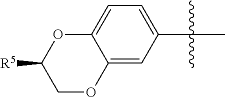 | 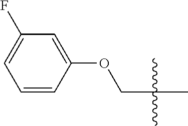 [SMILES: *COc1cccc(F)c1] | 5-i-indl | 1-oxo | (R, S) |
| 34 | | | | 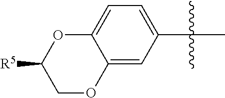 | 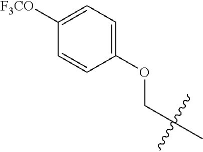 [SMILES: *COc1ccc(OC(F)(F)F)cc1] | 5-i-indl | 1-oxo | (R, S) |
| 35 | | | | 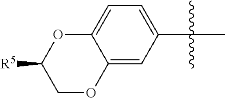 | 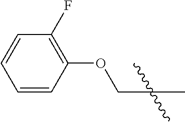 [SMILES: *COc1ccccc1F] | 5-i-indl | 1-oxo | (R, S) |
| 36 | | | | 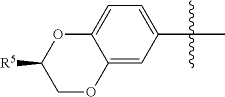 | 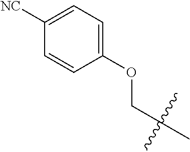 [SMILES: *COc1ccc(C#N)cc1] | 5-i-indl | 1-oxo | (R, S) |
| 37 | | | | 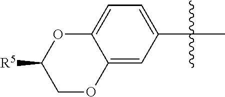 | 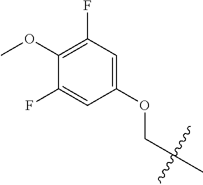 [SMILES: *COc1cc(F)c(OC)c(F)c1] | 5-i-indl | 1-oxo | (R, S) |
| 38 | | | | 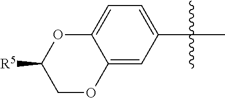 | 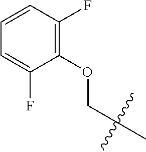 [SMILES: *COc1c(F)cccc1F] | 5-i-indl | 1-oxo | (R, S) |
| 39 | | | | 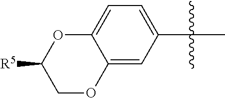 | 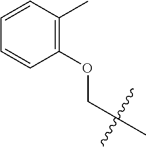 [SMILES: *COc1ccccc1C] | 5-i-indl | 1-oxo | (R, S) |
| 40 | | | | 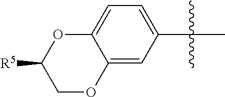 | 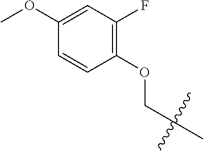 [SMILES: *COc1ccc(OC)cc1F] | 5-i-indl | 1-oxo | (R, S) |
| 41 | | | 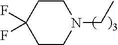 | | | Ph | 4-CONH₂ | (S) |
| 42 | | | 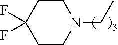 | | | 5-i-indl | 1-oxo | (S) |
| 43 | | | | 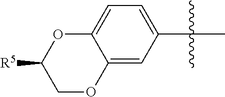 | 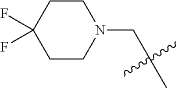 [SMILES: *CN1CCC(F)(F)CC1] | 5-i-indl | 1-oxo | (R, S) |
| 44 | | | | 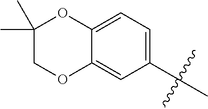 [SMILES: *c1ccc2c(c1)OCC(C)(C)O2] | | Ph | 4-CONH₂ | (S) |
| 45 | | | 4-c. PrO(CH₂)₃ | | | 5-i-indl | 1-oxo | (S) |
| 46 | | | 4-c. PrO(CH₂)₃ | | | Ph | 4-CONH₂ | (S) |

### EXPERIMENTAL

### Abbreviations

1°, primary; 4°, quaternary; Ar, aromatic ring; Boc, tert-butylcarbonate; Boc₂O, di-tert-butyl dicarboxylate; br, broad; brine, saturated sodium chloride solution; C, carbon; cAMP, cyclic adenosine monophosphate; CDCl₃, deuterated chloroform; COMFA, comparative molecular field analysis; COSY, correlation spectroscopy; d, doublet; D₂O, deuterated water; DCC, dicyclohexylcarbodiimide; DCM, dichloromethane; dd, doublet of doublets; DEAD, diethyl azodicarboxylate; def, deformation; DEPT, distortionless enhanced polarisation transfer; DMF, N,N-dimethylformamide; DMSO, dimethyl sulphoxide; DMSO-d, deuterated dimethyl sulphoxide; DPPA, Diphenylphosphoryl azide; dt, doublet of triplets; EDC, 1-ethyl-3-(3-dimethylaminopropyl) carbodiimide hydrochloride; eq, molar equivalents; ES, electrospray; Et₂O, diethyl ether; EtOAc, ethyl acetate; EtOH, ethanol FA, formic acid; FT-IR, fourier transform-Infrared H₂ hydrogen gas; HCl, hydrochloric acid; HMBC, heteronuclear multiple bond correlation; HPLC, high performance liquid chromatography; HSQC, heteronuclear single quantum correlation; J, Coupling constant; J_{CF}, Carbon-Fluorine coupling constant; K₂CO₃, Potassium carbonate; KHSO₄, potassium hydrogen sulfonate; KMnO₄, potassium permanganate; lit, literature; m, multiplet; MeCN, acetonitrile; MeOH, methanol; MgSO₄, anhydrous magnesium sulphate; Mp, melting point; MS, mass spectrometry; MW, microwave; m/z, observed ion; NaH, sodium hydride; NaHCO₃, Sodium Hydrogen Carbonate; NaOH, sodium hydroxide; NH₃, Aqueous ammonia solution (35%); Na₂504, anhydrous sodium sulphate; NMR, nuclear magnetic resonance spectroscopy; Pd, palladium; PDE, phosphodiesterase; phth, phthalimide; PLC, preparative layer chromatography; PMA, phosphomolybdic acid; ppm, parts per million; PPTS, pyridinium para-tolueunesulphonate; ^{c}Pe, cyclopentyl; ^{c}Pr, cyclopropyl; para-toluene surfonylchloride; q, quadruplet; Rₜ, retention time s, singlet str, stretch; t, triplet; TEA, triethylamine; TFA, trifluoroacetic acid; THF, tetrahydrofuran; THP, tetrahydropyran; TMS, tetramethylsilane; TOF, time of flight.

### General Chemistry

Chemicals and solvents were purchased from standard suppliers and used without further purification. Merck Kieselgel 60, 230-400 mesh, for flash column chromatography was supplied by Merck KgaA (Darmstadt, Germany) and deuterated solvents were purchased from Goss International Limited (England) and Sigma-Aldrich Company Ltd (England).

Unless otherwise stated, reactions were earned out at ambient temperature. Reactions were monitored by thin layer chromatography on commercially available precoated aluminium backed plates (Merck Kieselgel 60 F₂₅₄). Visualisation was by examination under UV light (254 and 366 nm). General staining carried out with Ninhydrin, KMnO₄ or PMA, or LC MS (gradient described bellow)

All organic extracts after aqueous work-up procedures were dried over MgSO₄ or Na₂SO₄ before filtering and evaporation to dryness. Organic solvents were evaporated under reduced pressure at ≦40′C (water bath temperature). Purification using preparative layer chromatography was carried out using Fluka silica gel 60 PF₂₅₄ containing gypsum (200 mm×200 mm×1 mm). Flash chromatography was performed using Merck Kieselgel 60 (0.040-0.063 mm) in classical glass columns or using a solvent gradient on Flashmaster or Isolera 4. For dry loading, the samples were adsorbed on bulk Isolute® available from Biotage.

Melting points were recorded on a Electrothermal melting point apparatus or Mettler Toledo Melting Point System MP50 and were uncorrected.

FT-IR spectra were recorded as thin films or KBr discs in the range of 4000-500 cm⁻¹ using and Avatar 360 Nicolet FT-IR spectrophotometer. Optical rotation was measured on a Bellingham-Stanley ADP220 polarimeter.

Mass spectra (TOF ES +/−) were recorded on a Waters 2795 separation module/micromass LCT platform.

¹H NMR spectra were recorded on a Bruker-AV 400 at 400.13 MHz. ¹³C NMR spectra were recorded at 101.62 MHz. Chemical shifts (δ) are recorded in ppm with reference to the chemical shift of the deuterated solvent/an internal TMS standard. Coupling constants (J) are recorded in Hz and the significant multiplicites described by singlet (s), doublet (d), triplet (t), quadruplet (q), broad (br), multiplet (m), doublet of doublets (dd), doublet of triplets (dt). Spectra were assigned using appropriate COSY, DEPT, HSQC and HMBC sequences. Unless otherwise stated all spectra were recorded in CDCl₃.

Analytical HPLC were performed on a Shimadzu UFLCXR system coupled to an Applied Biosystems API2000. Two columns thermostated at 40° C. were used.

Column one: Gemini-NX 3u-110A; 50×2 mm

### Column two: Luna 3u (PFP2) 110A, 50×2

Flow rate 0.5 ml/min. UV detection at 220 and 254 nm.
Gradient: Pre-equilibration run for one min at 10% 8, 10 to 98% solvent B in 2 min, 96% for 2 min, 98 to 10% B in 0.5 min then 10% for one min
Solvent A: 0.1% Formic Add in water; solvent B; 0.1% Formic Acid in MeCN.

### Reference Examples

### Synthesis of Intermediates

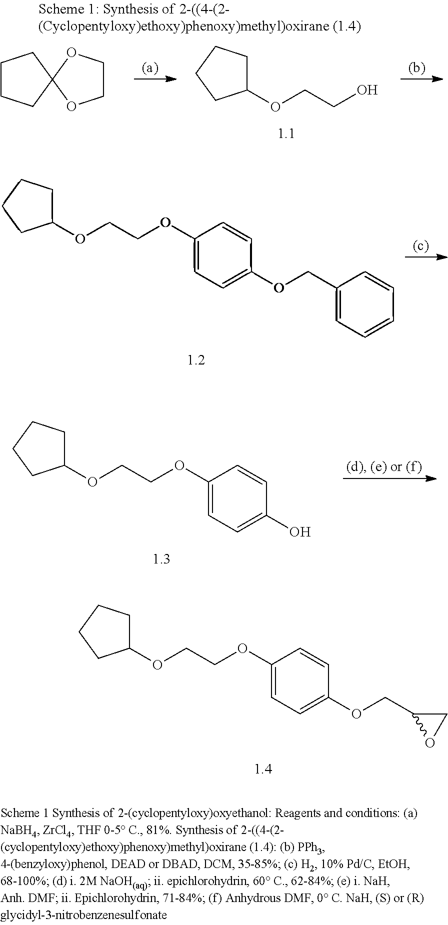

Step (a): 2-(cyclopentyloxy)oxyethanol (1.1) was synthetised according to Synthetic Communications, 29:8, 1257-1261;
Step (b): 2-(cyclopentyloxy)oxyethanol (1.1) was coupled to 4-(Benzyloxy)phenol under standard Mitsunobu conditions to afford (1.2).
Step (c): Hydrogenation of (1.2) with Pd/C in EtOH affords (1.3) in quantitative yield,
Step (e): NaH 60% suspension in mineral oil (863 mg, equivalent to 518 mg of NaH, 21.58 mmol. 1.1 eq) was suspended in dry DMF (20 mL) with stirring under a nitrogen atmosphere. After 5 minutes, (1.3) (4.360 g, 19.61 mmol) in dry DMF (20 mL) was added dropwise with the vessel cooled over an ice bath. This was then allowed to stir at rt for 20 minutes before addition of epichlorohydrin (15.34 mL, 196.10 mmol, 10 eq). The mixture was stirred for 7 h then quenched cautiously with MeOH. After removal of all volatiles, the crude residue was partitioned between water (30 mL) and Et2O (30 mL) and the aqueous layer washed again with Et2O (3×30 mL). The combined organic extracts were concentrated before purification over a silica plug (initial wash with hexanes, followed by EtOH/DCM 5:95) to give 4.558 g of clear yellow oil.
Step (f): The enantiomerically pure (1.4) (R or S) is prepared following the procedure described in Eur. J. Med. Chem., 37, 2002, 731-741.

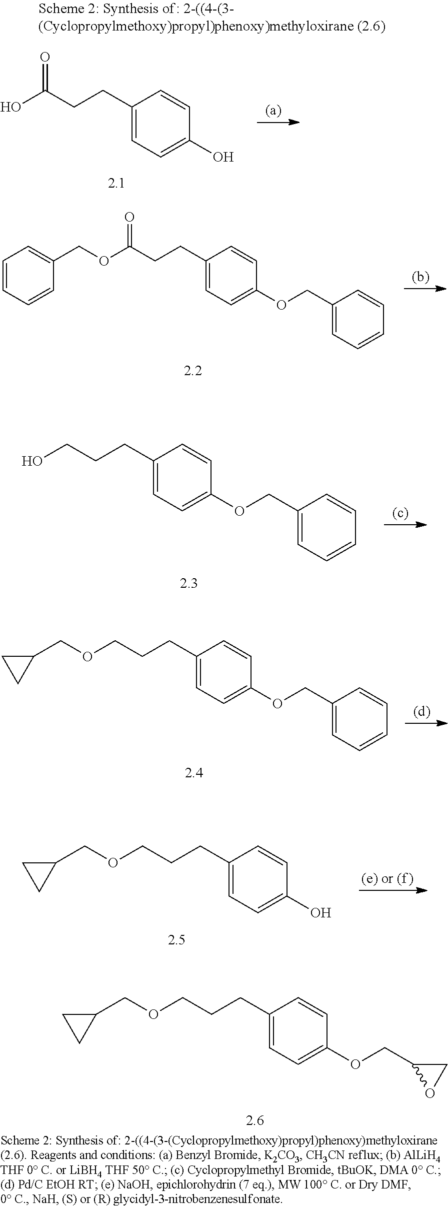 [SMILES: O=C(O)CCc1ccc(O)cc1 ; O=C(CCc1ccc(OCc2ccccc2)cc1)OCc1ccccc1 ; OCCCc1ccc(OCc2ccccc2)cc1 ; c1ccc(COc2ccc(CCCOCC3CC3)cc2)cc1 ; Oc1ccc(CCCOCC2CC2)cc1 ; c1cc(OCC2CO2)ccc1CCCOCC1CC1]

Step (a); (b): Compound (2.3) is commercially available or can be synthesized using standard protocols from the commercially available 3-(4-hydroxyphenyl) propanoic acid (acid (2.1)) as outlined in scheme 2.
Step (c): Alkylation of (2.3) following the procedure described in Tetrahedron, 2007, 63, 1872-1876, affords (2.4)
Step (d): Hydrogenation with Pd/C in EtOH affords (2.5).
Step (e): The racemic (2.6) is obtained by alkylation of (2.5) with excess epichlorohydrin in presence of NaOH sad under MW heating.
Step (f): The enantiomerically pure (2.6) or 5) is prepared following the procedure described in Eur. J. Med. Chem., 37, 2002, 731-741

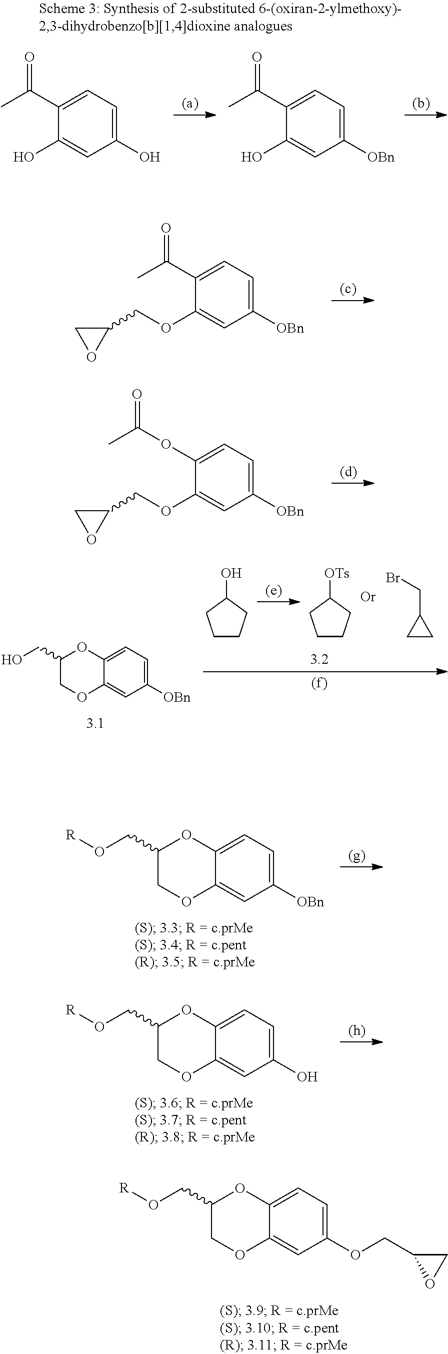

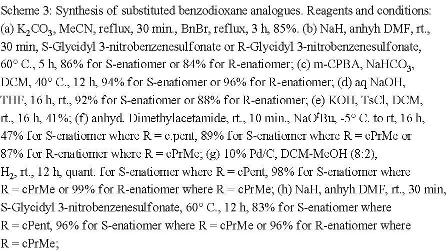

The synthesis of (3.1) was adapted from J. Agric. Food Chem. 2002, 50, 4554-4566 with:

Step (a): Bioorg. Med. Chem. Lett., 18 (2008), 5415-5419;
Step (b): (R) and (S) Glycidyl 3-nitrobenzenesulfonate were used instead of epichlorhydrin using conditions adapted from Eur. J. Med. Chem. 37, 2002, 731-741;
Step (c): Bayer Villiger oxidation following the protocol described in Synthetic Communications, 39: 20 (2009), 3693-3709.
Step (d): One-pot saponification-cyclisation (from the original paper).
Step (e): Synthesis of (3.2) was adapted from WO 2009/062990 02.
Step (f): Synthesis of (3.3) (3.5) was adapted from Tetrahedron, 63, 2007, 1872-1876
Step (g): Hydrogenation with Pd/C in DCM MeOH, rt, O/N.
Step (h): Synthesis of (3.9) (3.11) was adapted from Eur. J. Med. Chem. 37, 2002, 731-741.

The Right Hand Side phenoxyethers were either commercially available (parafluoro and paramethoxy derivatives) as free base or as a salt or were synthesised accordingly following the protocols described below:

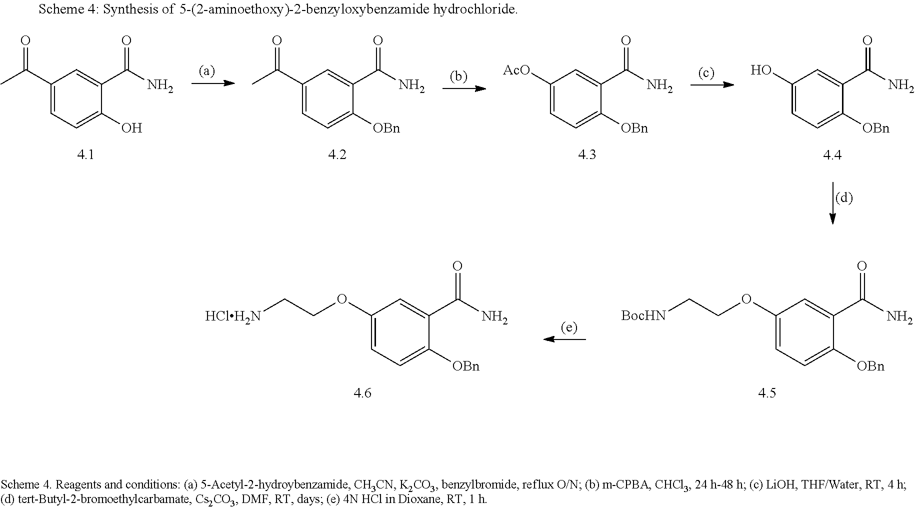

Step (a): 5-Acetyl-2-hydroxybenzamide (4.1) (8.2 g, 45.76 mmol), K₂CO₃ (9.488 g, 68.65 mmol, 1.5 eq) and BnBr (8.609 g, 5.987 mL, 50.34 mmol, 1.1 eq) were heated under reflux in MeCN (100 mL) overnight. The mixture remained a white suspension throughout. After removal of MeCN under reduced pressure, the crude product was dispersed in water (100 mL), and extraction with EtOAc (50 mL) was attempted. The white precipitate was found to be insoluble in either layer, and so was collected by filtration (vacuum) to give 9.41 g of white solid. The aqueous layer was separated in the filtrate and washed with a further 50 mL of EtOAc. Concentration of the combined organic layers gave a white solid with an odour of BnBr. This was sonicated in PE, before filtering, and washing with a small amount of DCM/PE 1:1. The total recovered yield of product (4.2) is 12.077 g (98%) Step (b); 5-Acetyl-2-benzyloxybenzamide (4.2) (5.00 g, 18.60 mmol) was dispersed in chloroform (50 mL) to give a white suspension which cleared upon addition of m-CPBA 70-75% in water (6.86 g, 27.8 mmol, equivalent to 4.806 g). The mixture was stirred at room temperature for 24 hours, at which time LCMS analysis indicated partial conversion had taken place. A further 0.5 eq of m-CPBA were added and stirring was continued for a further 48 hours. The mixture was diluted with DCM (50 mL) before washing with sat. aq. NaHCO₃ (50 mL). The aqueous layer was washed with further DCM (2×50 mL) and the combined organic layers washed once more with NaHCO₃ (50 mL). The organic layer formed a cloudy white suspension. This was solubilised by addition of a little MeOH, followed by drying with sodium sulphate. Concentration gave 7.1 g of a pale yellow oil. Subsequently, the crude oil was dissolved in the minimum amount of EtOAc, before adding PE cautiously to cause precipitation of a white solid (4.3), which was collected by filtration (vacuum) and washed further with PE. Yield: 4.133 g, 78%.
Step (c): 4-benzyloxy-3-carbamoylphenyl acetate (4.3) (4.1 g. 14.5 mmol) and LiOH.H₂O (1.5 eq., 21.6 mmol, 900 mg) were dissolved in and mixture THF/Water (25125 ml) to form a yellow suspension, which darkens quickly. The mixture is stirred at room temperature for 4 h. THF is removed under reduced pressure and the remaining aqueous slurry is diluted with 2N NaOH (25 mL) to form a complete solution. This was washed with AcOEt (25 mL). The basic aqueous layer was then acidified with concentrated HCl before extraction with AcOEt (3×30 mL). The combined organic phase were washed with brine, dried over Na₂SO₄, filtered and evaporated to give a brown solid (100% crude yield). The brown solid (4.4) can be further purified by re-crystallization from MeOH/Et2O or by FCC using a gradient DCM/AcOEt 0 to 100%.
Step (d): The ether (4.5) can be synthesized using the Mitsunobu reaction protocol but the yield and purity were unsatisfactory. An alternative, new general method by alkylation is described below.

### General Procedure for Phenol Alkylation

2-benzyloxy-5-hydroxybenzamide (4.4) (1.0 g. 4.1 mmol) and Cs₂CO₃ (1.1 eq 4.5 mmol, 1.5 g) were dispersed in DMF (50 mL) to give a white suspension, which was stirred for 30 minutes at room temperature. tert-Butyl-2-bromoethylcarbamate 4.5 mmol, 1 g.) was added in portions, and the mixture stirred at room temperature. The reaction was monitored by TLC or HPLC every 24 h and further aliquots (0.25 eq.) of Cs₂CO₃ or K₂CO₃ (at once) and tert-Butyl-2-bromoethylcarbamate (portion wise) were added. The reaction can require several aliquots and several days for completion or satisfactory conversion. When the reaction is complete the DMF is evaporated. Water added to the residue is extracted with AcOEt. The aqueous phase is further washed with AcOEt (2×). The organic phases are combined, further washed with 2N NaOH, brine, dried over Na₂SO₄, filtered and evaporated to afford (4.5).

Depending on the example of NH-Boc protected ether, some are pure enough to be used without further purification. Other requires purification which can be achieved by FCC on silica,

### Step (e) General Method for the Deprotection of NH-Boc Ether Derivatives.

tert-Butyl-2-(4-benzyloxy-3-carbamoylphenoxy)ethylcarbamate (2 g) (4.5) is dissolved in 4N HCl in dioxane (50-100 ml) and stirred at room temperature. The initial orange solution becomes a suspension after a few minutes of stirring. After one hour, the suspension is concentrated under vacuum and excess Et₂O is added to precipitate a white solid, which is filtered, further washed with Et₂O and dried under vacuum to afford 5-(2-aminoethoxy)-2-benzyloxybenzamide hydrochloride (4.6) (quantitative yield).

### Variant Procedure: General Procedure for NH-Boc Deprotection of Amines

The NH-Boc-protected amine was dissolved or dispersed in DCM (approximately 20 mL/g of compound) and stirred at room temperature for 2 minutes. 4M HCl/1,4-dioxane (approximately 20 mL/g of compound) was added, and the mixture stirred at room temperature for 1 hour. Excess petroleum ether 40-60 was added to the mixture and the resultant precipitate collected by filtration, and was further with petroleum ether 40-60,

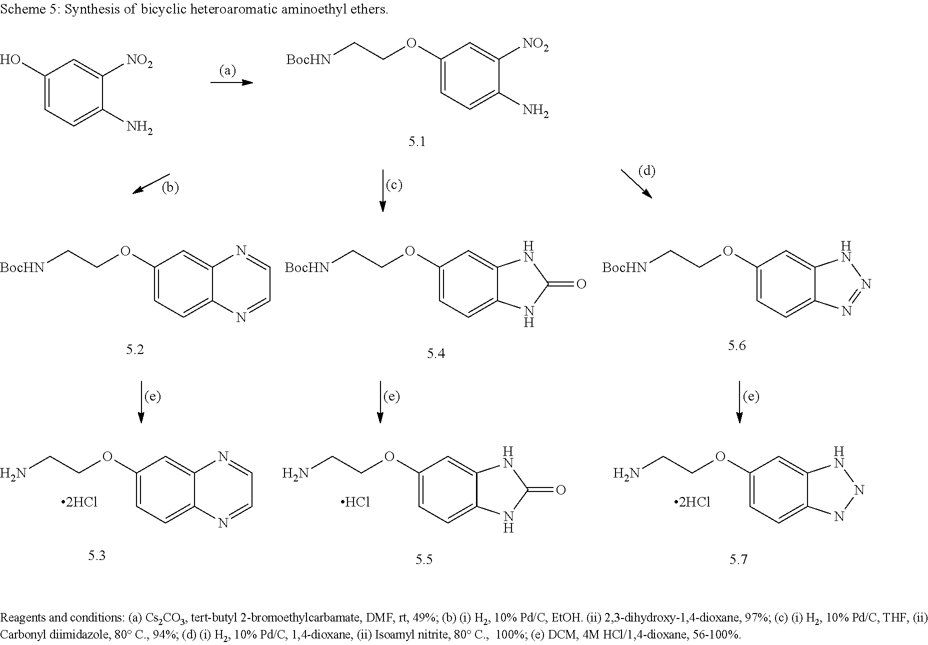

### tert-Butyl-2-(4-amino-3-nitrophenoxy)ethylcarbamate (5.1)

(5.1) was synthetised from the commercially available 4-Amino-3-nitrophenol (5 g) following the general procedure I. After extraction, further purification was carried out by FCC (gradient in EtOAc/petroleum ether 40-60) to give: 4.7499 of bright orange solid (49%).

### tert-Butyl-2-(quinoxalin-6-yloxy)ethylcarbamate (5.2)

tert-Butyl-2-(4-amino-3-nitrophenoxy)ethylcarbamate (5.1) (1.00 g, 3, 36 mmol) was dissolved in EtOH (20 mL) and the flask flushed with nitrogen, before adding 10% Pd/C (100 mg). The solution was degassed and stirred under an hydrogen atmosphere (balloon) at room temperature for 6 h. TLC analysis (EtOAc) indicated the nitro compound had been fully reduced. The flask was evacuated and filled with nitrogen several times, before addition of 2,3-dihydroxy-1,4-dioxane (484 mg, 4.03 mmol, 1.2 eq). After overnight stirring at room temperature, TLC analysis (EtOAc/PE 8:2) indicated disappearance of the intermediate phenylenediamine. The mixture was filtered over a bed of celite with washings of MeOH and concentrated to give a brown oil. This was dispersed in water (50 mL) and extracted with DCM (3×30 mL). The combined organic layers were concentrated to give 1.139 g of dark brown oil, which slowly crystallised overnight (quantitative). No further purification was necessary.

### tert-Butyl-2-(2-oxo-2,3-dihydro-1H-benzo[d]imidazol-5-yloxy)ethylcarbamate (5.4)

tert-Butyl-2-(4-amino-3-nitrophenoxy)ethylcarbamate (5.1) (1.00 g, 3.36 mmol) as dissolved in dry THF (20 mL) and 10% Pd/C (100 mg) added. The mixture was hydrogenated overnight. After a total of 29 hours of hydrogenation, the flask was evacuated and filled with nitrogen several times, before adding carbonyl diimidazole (672 mg, 4.14 mmol, 1.2 eq), and heating at 80° C. overnight. TLC analysis indicated disappearance of the intermediate phenylenediamine, so the mixture was passed through a bed of celite with washings of MeOH, before concentrating. The residue was dispersed in EtOAc (50 mL, only partially soluble) and washed with half-saturated aq. NH₄Cl (2×50 mL), then brine (50 mL). The organic layer was dried over Na₂SO₄ before concentrating to give 474 mg of pale yellow solid. The low yield prompted investigation of the Na₂SO₄, which was dispersed in MeOH, and the supernatant analysed by TLC. The product was found to have limited solubility in EtOAc and to have crashed out and deposited with the drying agent. Subsequently, the sodium sulphate was washed with MeOH over a sinter, and the filtrate concentrated, giving a further 842 mg of beige solid. This was combined with the original collected solid, and purified further by FCC (gradient in MeOH/DCM) During FCC purification, the compound precipitated from the column, causing blockage of the frit. Pure fractions were isolated, and the column dismantled, and the silica washed with MeOH/DCM (1:9). The washing filtrate was concentrated, and washed with Et₂O, leaving behind the desired compound as a brown solid, 932 mg (94%).

### tert-butyl-2-(1H-benzo[d][1,2,3]triazol-6-yloxy)ethylcarbamate (5.6)

tert-Butyl-2-(4-amino-3-nitrophenoxy)ethylcarbamate (5.1) (1.00 g, 3.36 mmol) was dissolved in dry 1,4-dioxane (20 mL) and 10% Pd/C (100 mg) added. The mixture was hydrogenated overnight. After a total of 29 hours, the flask was evacuated and filled with nitrogen several times. Isoamyl nitrite (0.495 mL, 3.70 mmol, 1.1 eq) was added and the mixture was heated at 80° C. overnight. TLC analysis (MeOH/DCM 1:9) indicated complete conversion. The mixture was passed through a bed of celite, with washings of MeOH. After concentration of the filtrate, the resulting residue was dissolved in EtOAc (50 mL) and washed with water (50 mL) and brine (50 mL). The organic layer was then concentrated on a rotary evaporator with heating under high vacuum to remove the remaining traces of isoamyl alcohol (bp 132° C.), giving 973 mg of brown oil which slowly crystallised overnight (quantitative).

### 2-(Quinoxalin-6-yloxy)ethanamine dihydrochloride (5.3)

The title compound was prepared from tert-Butyl-2-(quinoxalin-6-yloxy)ethylcarbamate (5.2) according to the general procedure for N-Boc deprotection of amines.

### 5-(2-Aminoethoxy)-1H-benzo[d]imidazol-2(3H)-one (5.5)

The title compound was prepared from ten-Butyl-2-(2-oxo-2,3-dihydro-1,1-benzo[d]imidazol-5-yloxy)ethylcarbamate (5.4) according to the general procedure for N-Boc deprotection of amines.

### 2-(1H-Benzo[d][1,2,3]triazol-6-yloxy)ethanamine dihydrochloride (5.7)

The title compound was prepared from tert-butyl-2-(1H-benzo[d][1,2,3]triazol-6-yloxy)ethylcarbamate (5.6) according to the general procedure for N-Boo deprotection of amines.

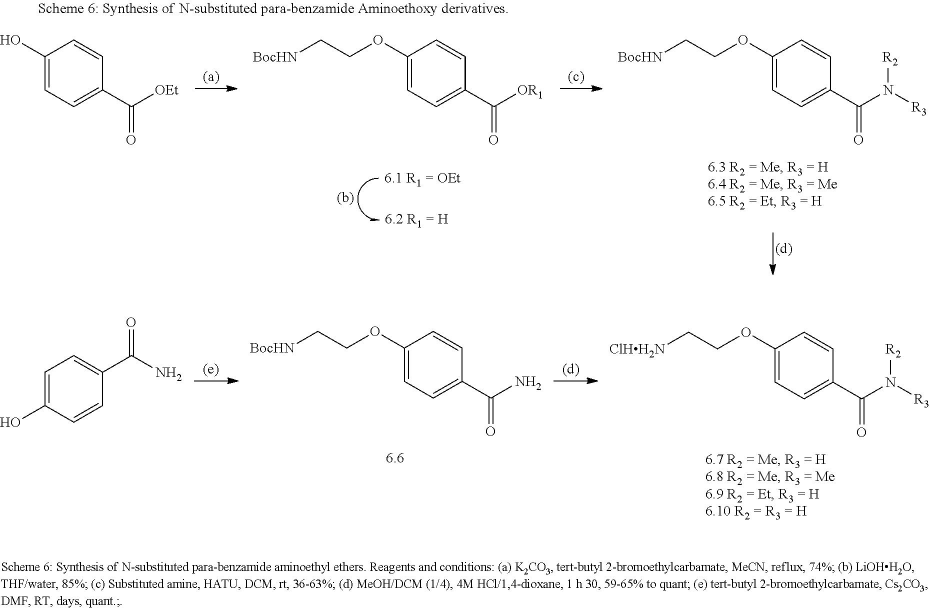

### Ethyl 442-(tert-butoxycarbonylamino)ethoxy)benzoate (6.1)

(6.1) can be prepared following the conditions described in the scheme from commercially available Ethyl 4-hydroxybenzoate (3.371 g, 20.28 mmol). The crude product was further purified by FCC (gradient in acetone/petroleum ether 40-60) to give 4.622 g of colourless oil (74%). Or (61) can be prepared following the general procedure for phenol alkylation I (99% crude yield).

### 4-(2-(tert-Butoxycarbonylamino)ethoxy)benzoic acid (6.2)

Ethyl-4-(2-(tert-butoxycarbonylamino)ethoxy)benzoate (6.1) (3.586 g, 12.14 mmol) was dissolved in THF/water (1:1, 80 mL) and the flask flushed with nitrogen gas. LiOH.H₂O (973 mg, 23.18 mmol, 1.9 eq) was added, causing the mixture to turn from a clear colourless solution to a white suspension. The mixture was stirred at room temperature overnight. LCMS analysis indicated slow reaction progression. LiOH.H₂O (2 eq) was added and stirring continued for a further overnight period. After a total of 6 nights, the reaction was nearly complete. LiOH.H₂O (1 eq) was added and stirring continued at room temperature overnight. The reaction failed to progress any further, so the mixture was concentrated under reduced pressure, leaving aqueous solution. This was diluted with water (40 mL), before washing with DCM (50 L). However this caused formation of an emulsion. Et₂O (200 mL) was added to separate the layers, and the organic layers discarded. The aqueous layer was acidified to pH5 with aq. 2M HCl, causing a white precipitate to form. This was collected by filtration (vacuum) and washed with water before drying to give 2.919 g of white amorphous solid (85%).

### General Procedure for HATU Amide Coupling:

4-(2-(tert-Butoxycarbonylamino)ethoxy)benzoic acid (6.2) and HATU (1.041 g, 2.74 mmol, 1.1 eq) were dissolved in DCM (10 mL). To this stirred solution was added the appropriate amine solution (either 2M MeNH₂ in THF, 2M Me₂NH in MeOH or 2M EtNH₂ in THF; 12.44 mmol, 5 eq), and the mixture left to stir overnight at room temperature. The mixture was concentrated and dispersed in EtOAc (50 mL), before washing with aq. sat. NH₄Cl (50 mL), aq. sat. NaHCO₃ (50 mL), water (50 mL) and brine (50 mL). The crude mixtures were further purified by FCC.

### tert-Butyl-2-(4-(methylcarbamoyl)phenoxy)ethylcarbamate (6.3)

The title compound was synthesised from 4-(2-(tert-butoxycarbonylamino)ethoxy)benzoic acid (6.2) according to the general procedure for HATU amide coupling to give a white solid (267 mg, 36%).

### tert-Butyl 2-(4-(d methylcarbamoyl)phenoxy)ethyl carbamate (6.4)

The title compound was synthesised from 4-(2-(tert-butoxycarbonylamino)ethoxy)benzoic acid (6.2) according to the general procedure for HATU amide coupling to give a colourless solid (486 mg, 63%).

### tert-Butyl 2-(4-(ethylcarbamoyl)phenoxy)ethylcarbamate (6.5)

The title compound was synthesised from 4-(2-(tert-butoxycarbonylamino)ethoxy)benzoic acid (6.2) according to the general procedure for HATU amide coupling to give an off-white crystalline solid (432 mg, 56%),

### tert-Butyl 2-(4-carbamoylphenoxy)ethylcarbamate (6.6)

(6.6) was synthetised from the commercially available, 4-Hydroxybenzamide (10.00 g) following the general procedure I. After completion, the solvent was removed under reduced pressure, before dispersing the solid in 1M NaOH (400 mL) with vigorous shaking. The undissolved matter was collected by filtration and washed with water, followed by PE before drying to give 21.1 g of off-white solid (quantitative).

### 4-(2-Aminoethoxy)-N-methylbenzamide hydrochloride (6.7)

The title compound was prepared from tert-butyl 2-(4-(methylcarbamoyl)phenoxy)ethylcarbamate (6.3) according to the general procedure for N-Boc deprotection of amines.

### 4-(2-Aminoethoxy)-N,N-dimethylbenzamide hydrochloride (6.8)

The title compound was prepared from tert-butyl 2-(4-(dimethylcarbamoyl)phenoxy)ethylcarbamate (6.4) according to the general procedure for NH-Boc deprotection of amines.

### 4-(2-Aminoethoxy)-N-ethylbenzamide hydrochloride (6.9)

The title compound was prepared from tert-butyl 2-(4-(ethylcarbamoyl)phenoxy)ethyl carbamate (6.5) according to the general procedure for N-Boc deprotection of amines.

### 4-(2-Aminoethoxy)-benzamide hydrochloride (6.10)

The title compound was prepared from tert-butyl 2-(4-carbamoylphenoxy)ethylcarbamate (6.6) according to the general procedure for N-Boc deprotection of amines,

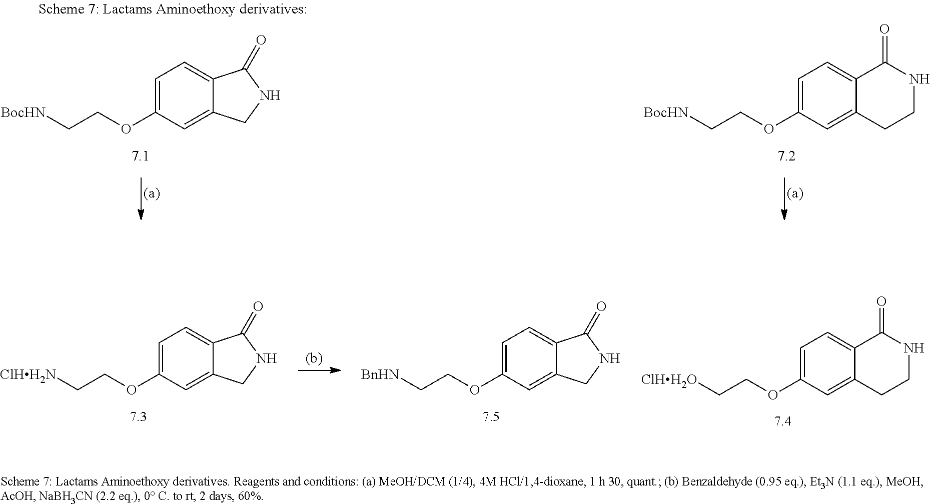

### tert-Butyl 2-(1-oxoisoindolin-5-yloxy)ethylcarbamate (7.1)

(7.1) was obtained by alkylation of the corresponding 5-hydroxyisoindolin-1-one. The 5-hydroxyisoindolin-1-one was synthesised from 4-methoxy-2-methylbenzoic acid in a similar manner to the protocol reported in international patent WO 2008/020306 A2. The initial esterification of 4-methoxy-2-methylbenzoic acid was carried out using SOCl₂ (3 eq) in place of sulphuric acid. In the following step, radical bromination proceeded using 1,1′-azobis(cyclohexanecarbonitrile) (0.1 eq) as the radical initiator, in place of benzoyl peroxide.

### tert-Butyl 2-(1-oxo-1,234-tetrahydrolsoquinolin-8-yloxy)ethylcarbamate (7.2)

(7.2) was obtained by alkylation of the corresponding 6-Hydroxy-3,4-dihydro-isoquinolin-1(2H)-one The 6-Hydroxy-3,4-dihydro-isoquinolin-1 (2H)-one was synthesised from 5-hydroxyindanone according to the procedure in WO 2007/001249 A1.

### 5-(2-aminoethoxy)isoindolin-1-one hydrochloride (7.3)

The title compound was prepared from (71) according to the general procedure for N-Boc deprotection of amines.

### 6-(2-aminoethoxy)-3,4-dihydroisoquinolin-1(2H)-one hydrochloride (7.4)

The title compound was prepared from (7.2) according to the general procedure for N-Boc deprotection of amines.

### 5-(2-(benzylamino)ethoxy)isoindolin-1-one (7.5)

The title compound was prepared by reductive alkylation between benzaldehyde and (7.3).

To a solution of (7.3) (18.8 mmol, 4.3 g, 1 eq) in anhydrous MeOH (100 mL) at CC was added TEA (2.895 mL, 20.68 mmol. 1.1 eq). The solution was stirred for 10 mm and benzaldehyde (1.82 mL, 17.86 mmol, 0.95 eq) was added with sufficient AcOH (˜0.2 mL) to maintain pH at −4.5. The mixture was stirred for 2 h at 0° C. and Sodium cyanoborohydride (3×867 mg; 41.37 mmol, 2.2 eq) was added in 3 portions over the period of 3 h, each time maintaining pH around 4.5. The mixture was then stirred over 2 days at RT. LCMS analysis showed formation of the target product and some bis-benzylated derivative.

The solvent was rotary evaporated under high vacuum to remove most of AcOH. The residue was re-dissolved in half saturated sodium bicarbonate solution and extracted with in DCM (50 mL). The aqueous phase was further extracted with DCM (2×50 mL). The organic layers were combined and washed with brine, dried over sodium sulphate, filtered and the solvent was rotary evaporated. The crude sample was purified by FCC using 4-6% 2M Ammonia in Methanol in DCM to yield 3.29 (60%) as a white solid. 1.69 (23%) of his benzylated derivative was also isolated.

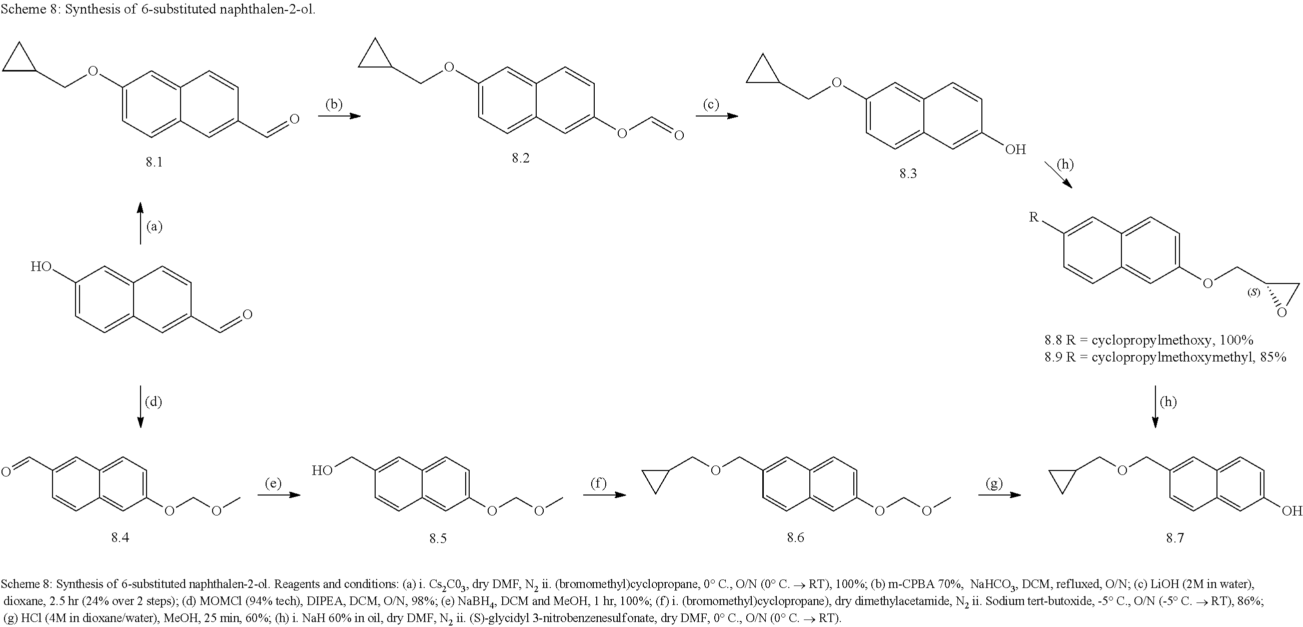

### Preparation of 642-cyclopropylmethoxy)-2-naphthaldehyde (8.1)

Commercially available, 6-hydroxy-2-naphthaldehyde (2.000 g, 11.6 mmol, 1 eq.) was dissolved in dry DMF (40 ml) under N₂ atmosphere and Cs₂CO₃ (1.1 eq.) was added. The solution was cooled to 0° C. and (Bromomethyl)cyclopropane (1.1 eq.) was added dropwise while stirring. The mixture was stirred O/N allowing the reaction temperature to ambient. After the reaction was completed, DMF was evaporated and the crude was quenched with saturated Na₂CO₃ and the aqueous phase was extracted with EtOAc twice. The organic layers were combined and washed with brine, dried over sodium sulphate, filtered and the solvent was rotary evaporated to give 2.589 g (100%) of an orange wax of acceptable purity.

### Preparation of 6-substituted naphthalen-2-yl formate (8.2) and 6-substituted naphthalen-2-ol (8.3)

In a two-neck round-bottom flask with a reflux condenser: m-CPBA 70% (2 eq.) and NaHCO₃ (7 eq.) were added with stirring to a solution of (8.1) (1 eq.) in DCM (50 ml). The mixture was refluxed and stirred O/N. The reaction was quenched with 2M HCl, and subsequently concentrated HCl. The aqueous phase was extracted with EtOAc twice. LCMS analysis of the crude (3.44 g) shows it is a mixture of formate (titled compound) and phenol (8.3). The crude was therefore used without purification.

### Preparation of 6-substituted naphthalen-2-ol (8.3)

To a stirred solution of crude (8.2) (3.44 g, 1.5 mmol, 1 eq.) in dioxane (40 ml), LiOH (2M in water, 2 eq.) was added. The mixture was stirred at room temperature for 2.5 hr and concentrated. The solution was quenched with 2M HCl (5 ml) and saturated NH₄Cl. The aqueous phase was extracted with EtOAc twice. The organic layers were combined and washed with brine, dried over sodium sulphate, filtered and the solvent was rotary evaporated. The crude was purified by FM (eluent PE/DCM 25 to 100% then followed with DCM/EtOAc 80/20%) to afford 0.775 g (24%) of a light green solid; mp: 126.5-129.6° C.

### Preparation of 6-(methoxymethoxy)-2-naphthaldehyde (8.4)

6-hydroxy-2-naphthaldehyde (4.000 g, 23.3 mmol, 1 eq.) and DIPEA (1.5 eq.) were dissolved in dry DCM (50 ml) and MOMCl (94% tech) (1.1 eq.) was added drop wise while stirring. The solution was stirred O/N. The reaction was quenched with saturated NH₄Cl and the aqueous phase was extracted with DCM twice. The organic layers were combined and washed with brine, dried over sodium sulphate, filtered and the solvent was rotary evaporated to give 4.960 g (98%) of a pale orange oil of satisfactory purity which solidifies to an off white solid (mp: 47-52° C.) upon standing at room temperature.

### Preparation of [6-(methoxymethoxy)naphthalene-2-yl]-methanal (8.5)

(8.4) (4.912 g, 22.7 mmol, 1 eq.) was dissolved in DCM (10 ml) and MeOH (90 ml). NaBH₄ (1.1 eq) was then added portion wise. The reaction mixture was stirred for 1 hr and then quenched with saturated NH₄Cl (after evaporation of MeOH and DCM) and the aqueous phase was extracted with DCM twice and lastly with EtOAc. The organic layers were combined and washed with brine, dried over sodium sulphate, filtered and the solvent was rotary evaporated to give 5.062 g (100%) of a pale orange solid of acceptable purity (mp: 52-54° C.).

### Preparation of 2-[(cyclopropylmethoxy)methyl]-6-(methoxymethoxy)naphthalene (8.6)

The title compound was prepared from alcohol (8.5) (4.9 g, 22.5 mmol, 1 eq.) in a similar procedure to the synthesis of intermediate (3.5), Crude (8.6) was purified using FCC (eluent Pet Ether/DCM 25-100%, followed with DCM/EtOAc 20%) to afford 5.280 g (86%) as a yellow oil.

### Preparation of 6-[(cyclopropylmethoxy)methyl]naphthalen-2-ol (8.7)

To a stirred solution of (8.6) (5.28 g. 19.4 mmol, 1 eq.) in MeOH (20 mL), HCl (4M in dioxane/water, 4 eq.) was added. The resulting mixture was stirred for 25 min. The mixture dioxane/methanol was evaporated. The resulting aqueous slurry was acidified with a little conc HCl and was extracted with EtOAc twice. The organic layers were combined and washed with brine, dried over sodium sulphate, filtered and the solvent was rotary evaporated to dryness. The crude was dry loaded with isolute, purified by FCC (eluent Pet Ether/DCM 25-100% followed with DCM/EtOAc 20%) to afford 2.670 g (60%) of a white solid (mp: 129-132° C.).

### Preparation of (2S)-2-([(6-substituted naphthalen-2-0)oxy]methyl)oxirane (8.8 and 8.9)

The title compounds were prepared respectively from phenol (8.3) (0.775 g, 3.5 mmol) and (8.7) (1.08 g, 3.7 mmol) in a similar procedure to the synthesis of intermediate (3.9) (except that the reaction proceeds at room temperature). After treatment, (8.8) was obtained as a light brown solid of acceptable purity (0.988 g, 100%). (8.9) was purified using FCC (eluent Pet Ether/DCM 25-100% followed with DCM/EtOAc 20%) to afford 1.146 g (85%) of colourless oil, which crystallized as white solid upon standing at room temperature (mp: 40-42° C.).

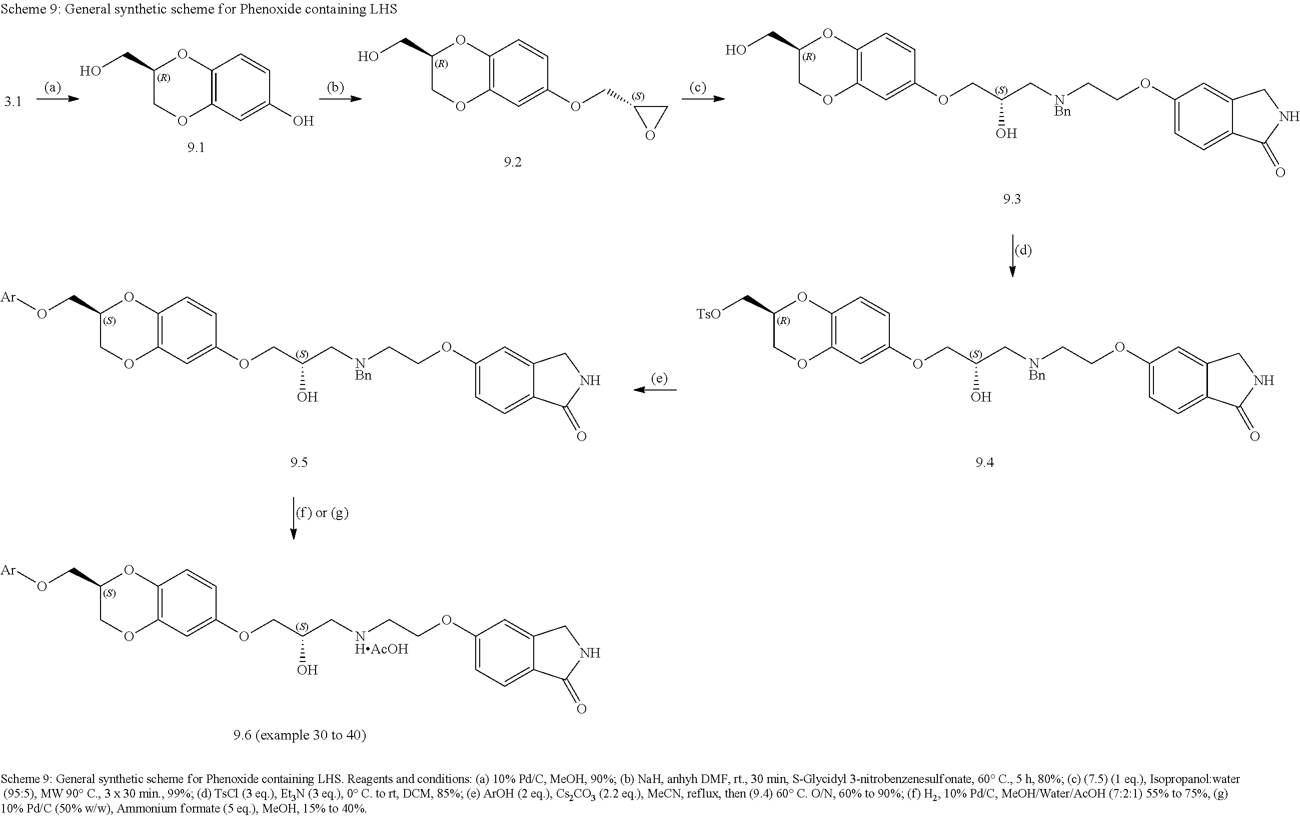

### (R)-2-(hydroxymethyl)-2,3-dihydrobenzo[b][1,4]dioxin-6-ol (9.1)

A suspension of Pd/C (4.1 g) in water (5 mL) was added to a round bottom flask containing (31) (153.14 mmol, 41.7 g) dissolved in MeOH (200 mL). This suspension was degassed and stirred at rt over 18 h under an hydrogen atmosphere. Completion of the reaction was monitored by LCMS/TLC. The suspension was then filtered through a bed of celite with some more MeOH (50 mL) washing. The filtrate was concentrated to get a crude product, which was dissolved in Ethyl acetate (150 mL) and washed with brine. Organic layer was then dried oven sodium sulphate and solvent was removed to afford (9.1) as a white solid (25 g, 90% yield).

### ((R)-6-((S)-oxiran-2-ylmethoxy)-2,3-dihydrobenzo[b][1,4]dioxin-2-yl methanol (9.2)

Synthesis of (9.2) was adapted from Eur. J. Med. Chem. 37, 2002, 731-741,

### 5-(2-(benzyl((5)-2-hydroxy-3-((R)-2-(hydroxymethyl)-2,3-dihydrobenzo[b][1,4]-dioxin-6-yloxy)propyl)amino)ethoxy)isoindolin-1-one (9.3)

(7.5) (1.5 g, 5.3 mmol) and (9.2) (1.5 g, 6.4 mmol, 1.2 eq.) were dissolved in Isopropanol/water (9.5:0.5) (15 mL) in a 40 mL microwave vessel. The reaction was then heated by MW at 90° C. for 3×30 min.

The solvent was rotary evaporated under high vacuum and the crude was purified by Flash master using a gradient of DCM and 2N-Ammonia in Methanol to afford (9.3) (2.7 g, 98%) as a white solid.

### ((S)-6-((S)-3-(benzyl(2-(1-oxoisoindolin-5-yloxy)ethyl)amino)-2-hydroxypropoxy)-2,3-dihydrobenzo[b][1,4]-dioxin-2-yl)methyl) 4-methylbenzenesulfonate (9.4)

To a solution of (9.3) (1.5 g, 2.88 mmol) in DCM (20 ml) was added triethylamine (1179 μl, 8.64 mmol, 3 eq.) and cooled to 0° C. p-Toluenesulfonylchloride (1.648 g, 8.64 mmol, 3 eq.) was then added portion wise. The reaction temperature was allowed to reach room temperature and was stirred over weekend. The reaction was monitored by LCMS. The reaction was diluted with more DCM (50 mL) and the organic layer was washed with water (2×50 mL), sat sodium bicarbonate (2×50 mL), brine, dried oven Na₂SO₄, filtered and evaporated. The crude was purified FCC using a gradient 1N NH3 in MeOH/DCM to get (9.4) as a colorless thick oil (1.6 g, 83%).

### 5-(2-(((S)-3-(R)-2-(aryloxymethyl)-2,3-dihydrobenzo[b][1,4]-dioxin-6-yloxy)-2-hydroxypropyl)(benzyl)amino)ethoxy)isoindolin-1-one (9.5)

### General Procedure

To a solution of Phenol (0.44 mmol) in MeCN (15 ml) was added Cs₂CO₃ (159 mg, 0.49 mmol, 1.1 eq.) and the suspension was refluxed for 30 min. After cooling to rt, (9.4) (150 mg, 0.22 mmol, 0.5 eq.) was added. The reaction was stirred at 60° C. for 18 h and was followed by LCMS. The solvent was rotary evaporated and the crudes compounds were purified by FCC using a gradient (2M NH3-MeOH/DCM) solvent system to afford the pure compounds (9.5) in 60 to 90% yield, used without further purification.

### 5-(2-((S)-3-((R)-2-(aryloxymethyl)-2,3-dihydrobenzo[b][1,4]-dioxin-6-yloxy)-2-hydroxypropylamino)ethoxy)isoindolin-1-one (9.6)

### Examples 30 to 40

### General Method

To a solution of (9.5) (˜50 mg) in MeOH:AcOH:H2O (7:2:1, 10 mL) was added Pd/C (5 mg). This mixture was stirred for 18 under an hydrogen atmosphere. Completion of the reaction was monitored by LCMS/TLC. The suspension was then filtered through a bed of celite and washed with MeOH (10 mL). The filtrate was concentrated by rotary evaporator to get the acetate salts (9.6) in 40 to 75% yield.

The free base can be isolated as white solids by a quick chromatography on silica using a gradient (2N NH3-MeOH/DCM),

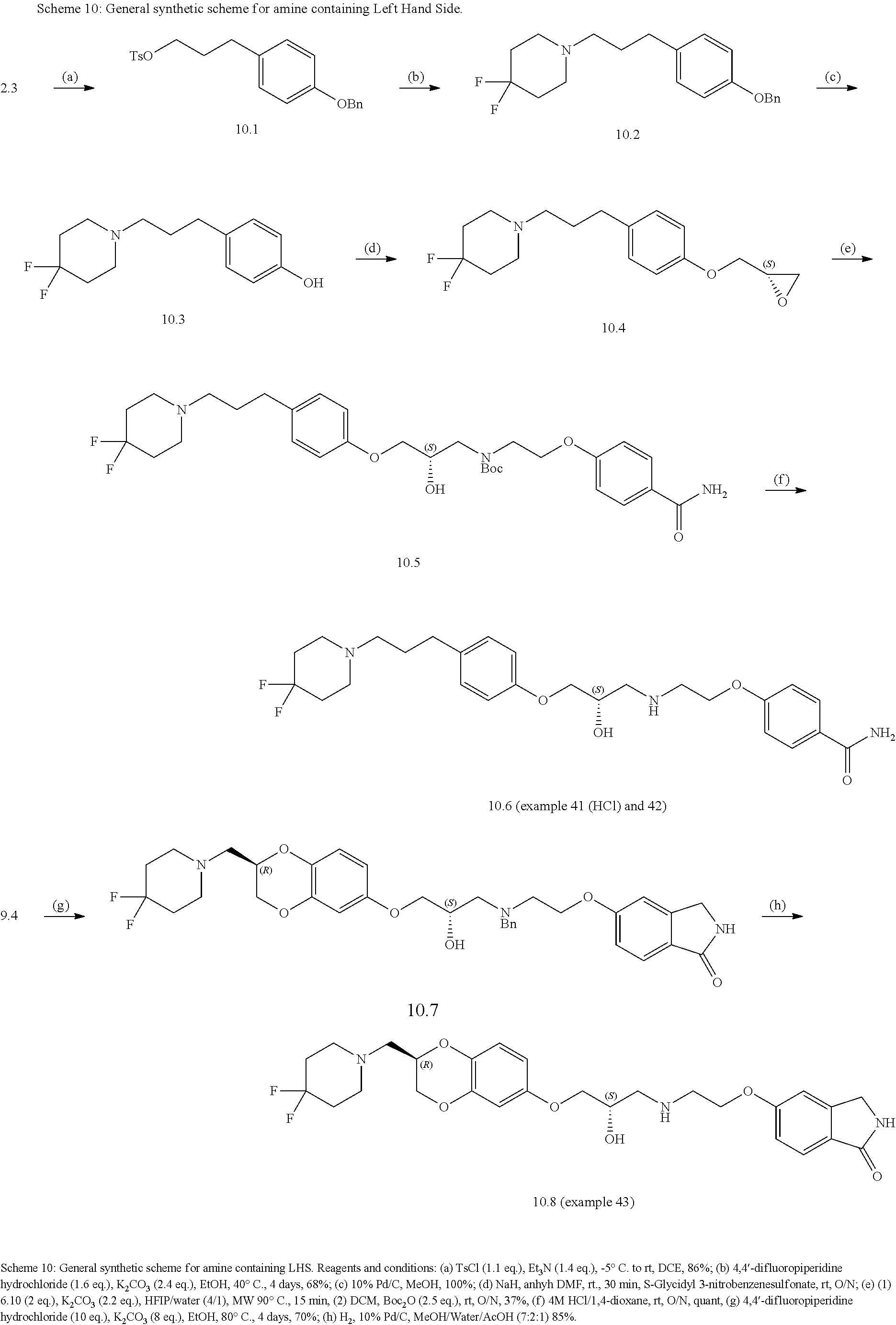

### 3-(4-(benzyloxy)phenyl)propyl 4-methylbenzenesulfonate (10.1)

To a solution of (2.3) (16.74 g, 69 mmol) and triethylamine (97 mmol, 13.5 mL, 1.4 eq.) in 500 mL DCE at −5° C. is added tosyl chloride (14.5 g, 76 mmol, 1.1 eq.), and the solution is allowed to warm up to rt O/N. The reaction mixture is partitioned between sat NH4Cl solution. The aqueous layer is washed with 3×DCM, and the organic layers are combined, washed with brine, dried over Na₂SO₄, filtered, and evaporated. The residue is purified by FCC (gradient Pet Ether to Etheyl Acetate), to afford the titled compound (23.575 g, 86%) as a white powder.

### 1-(3-(4-(benzyloxy)phenyl)propyl-4,4-difluoropiperidine (10.2)

Tosylate (10.1) (1.2 g, 3 mmol) is dissolved in EtOH (10 ml), potassium carbonate (1.2 eq., 3.6 mmol, 500 mg) and 4,4′-difluoropiperidine hydrochloride (1 eq., 3.3 mmol, 0.520 g) are added. The tosylate is not soluble in cold EtOH so the solution is stirred at 40° C. O/N. The reaction is monitored by LCMS. After ON, the reaction shows only a low conversion. So Difluoropiperidine hydrochloride (250 mg) (total 5 mmol, 1.6 eq.) and K₂CO₃ (500 mg) (total 7.25 mmol, 2.4 eq.) are added and the temp. is increased 50° C. for another 2 days. Ethanol is evaporated and the crude is extracted from sat NaHCO₃ with DCM and AcOEt. The organic phases are combined and washed with brine, dried over Na₂SO₄ filtered and evaporated. The purification is done on silica (30 g) and Isolera instrument using a gradient Pet Ether/DCM 50/50 to 100% DCM. The purification is followed by TLC (20% AcOEt/DCM, UV/KMnO4). The tubes with TM are combined and evaporated to afford (10.2) as a colourless oil (m=716 mg, 68%).

### 4-(3-(4,4-difluoropiperidin-1-yl)propyl)phenol (10.3)

(10.2) is dissolved in MeOH and Pd/C is added. The suspension is degassed and put under an H₂ atmosphere O/N. Filter the suspension over a celite bed and wash with a little methanol to get an orange oil (m=510 mg, 100%), used without further purification.

### (S)-4,4-difluoro-1-(3-(4-(oxiran-2-ylmethoxy)phenyl)propyl)piperidine (10.4)

(10.4) was synthetised according to Sharpless and Al., JOC, 54(6), 1989, 1295-1304. The crude ES a brownish oil used without further purification for next step.

### (S)-tert-butyl-2-(4-carbamoylphenoxy)ethyl (3-(4-(3-(4,4-difluoropiperidin-1-yl)propyl)phenoxy)-2-hydroxypropyl)carbamate (10.5)

(10.5) was prepared by coupling (10.4) (200 mg, 0.6 mmol) with (6.10) (280 mg, 1.2 mmol, 2 eq.), K₂CO₃ (195 mg, 1.4 mmol, 2.2 eq.) following the general procedure (ii) as described in the section “Examples” below. The secondary amine formed is then protected as a Boc group for purification purposes. The crude HFIP solution is diluted with DCM (5 ml) and Boc₂O (350 mg, 1.6 mmol, 2.5 eq.) a is added. The suspension is stirred at rt O/N. The reaction is monitored by LCMS. The reaction is completed after 2 days at rt. The crude is transferred onto isolute and evaporated thoroughly. The isolute is loaded on top of silica cartridge (15 g) and purified using Biotage Isolera with a gradient slow gradient DCM/AcOEt (100%). All fractions are analised by TLC (AcOEt Ninhydrin) Rf=0.1. The tubes with TM are combined and evaporated to get a white solid (m=140 mg, 36.8%).

### (S)-4-(2-(3-(4-(3-(4,4-difluoropiperidin-1-yl)propyl)phenoxy)-2-hydroxypropyl)amino)-ethoxy)benzamide dihydrochloride (10.6)

### Example 41

(10.5) (140 mg, 0.24 mmol) is dissolved in 4N HCl in dioxane (10 ml) and stirred at it, after on hour a white precipitate had appeared but the reaction is left O/N. The reaction is concentrated ether is added to facilitate a yellowish precipitate which is centrifuged and dried under vacuum to get an off white solid (m=125 mg, 100%).

### 5-(2-(benzyl((S)-3-((R)-2-((4,4-difluoropiperidin-1-yl)methyl)-2,3-dihydrobenzo[b][1,4]-dioxin-6-yloxy)-2-hydroxypropyl)amino)ethoxy)isoindolin-1-one (10.7)

(10.7) was synthetised in a similar manner to (10.2) from (9.4) (120 mg, 0.18 mmol) with potassium carbonate (221 mg, 1.6 mmol, 9 eq.) and 4,4′-difluoropiperidine hydro-chloride (280 mg, 1.8 mmol, 10 eq.) to afford after purification (10.7) as an off white solid (75 mg, 67%).

### 5-(2-((S)-3-((R)-2-((4,4-difluoropiperidin-1-yl)methyl)-2,3-dihydrobenzo[b][1,4]-dioxin-6-yloxy)-2-hydroxypropylamino)ethoxy)isoindolin-1-one (10.8)

### Example 43

(10.7) (75 mg, 0.12 mmol) was debenzylated by hydrogenation as described for examples (9.6) to afford (10.8) after purification on silica with a gradient 1N NH3 in MeOH/DCM, as a white solid (m=55 mg, 85%),

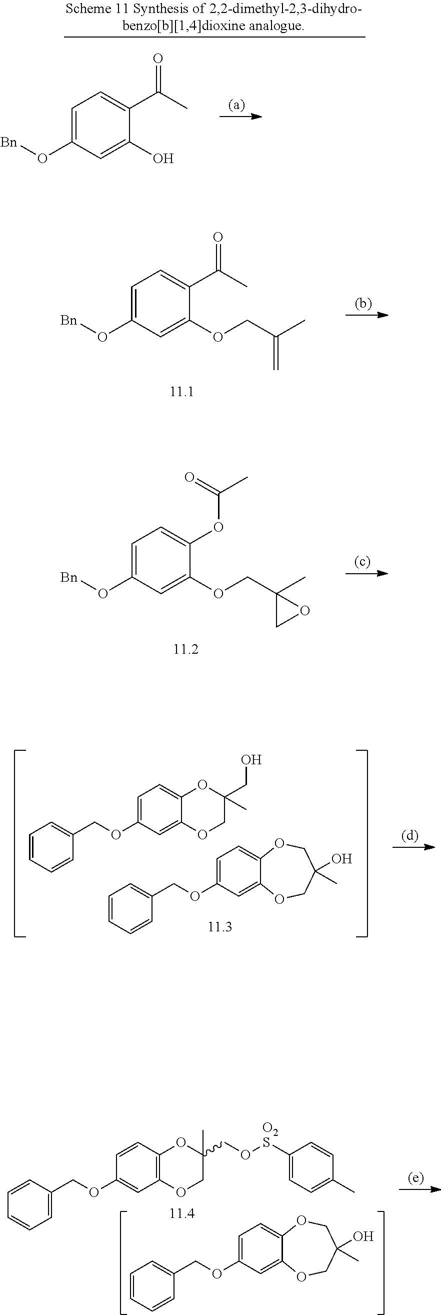

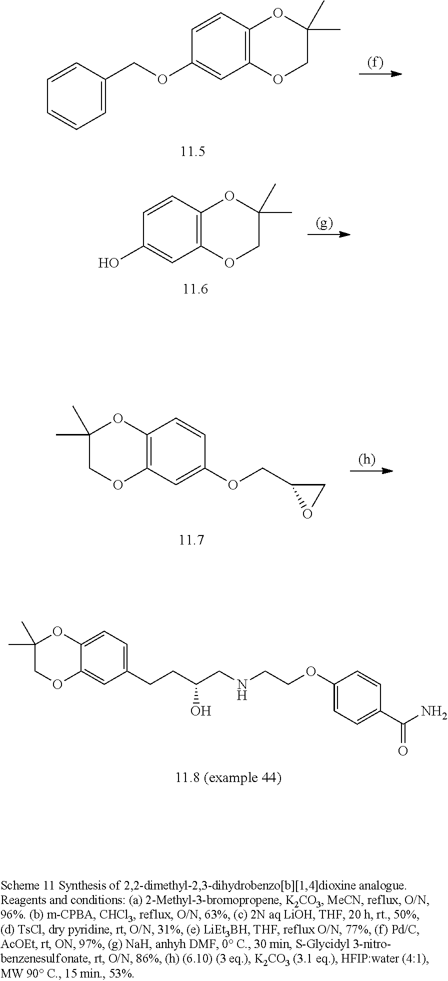

### 1-(4-(benzyloxy)-2-(2-methylallyloxy)phenylethanone (11.1)

The title compound was prepared by alkylation of the 4-benzyloxy-2-hydroxy-acetophenone (12 g, 49.5 mmol, 1 eq.) with 2-Methyl-3-bromopropene. The crude (11.1) was purified using FCC (eluent DCM/AcOEt) and followed by TLC (10% The tubes with TM are combined and evaporated to get a pale orangy oil. (m=14.2 g, 96%).

### 4-(benzyloxy)-2-(2-methyloxiran-2-yl)methoxy)phenyl acetate (11.2)

The procedure from J. Agric. Food Chem. 2002, 50, 4554-4566 with (11.1) to afford after purification (11.2) (8.2 g, 63%).

### 6-(benzyloxy)-2-methyl-2,3-dihydrobenzo[b][1,4]dioxin-2-yl)methanol and 7-(benzyloxy)-3-methyl-3,4-dihydro-2H-benzo[b][1,4]dioxepin-3-ol) (11.3)

To a stirred solution of crude (11.2) (6.69, 20 mmol) in THF (200 mL), was added aqueous 2M LiOH.H2O (1 g, 24 mmol. 12 mL). The solution was stirred at rt O/N. Completion of the reaction was monitored by TLC/LCMS.

A saturated solution of NH₄Cl (100 mL) was added to the solution and the THF phase was separated. The aqueous solution was further extracted with Ethyl Acetate (2×100 mL). The combined organic extract was washed with brine (100 mL), dried over sodium sulphate, filtered and solvent was evaporated under vacuo to obtained a crude yellow oil. Attempted purification failed on silica (gradient DCM/AcOEt; 0 to 50%) and alumina. The mix of alcohol (NMR analysis indicates that it is about a 50/50 mix) (recovered 2.9 g, 50%) is used without further purification as kinetic resolution is expected (primary alcohol being more reactive than tertiary!).

### 6-(benzyloxy)-2-methyl-2,3-dihydrobenzo[b][1,4]dioxin-2-yl)methyl 4-methylbenzene-sulfonate (11.4)

(11.3) (mix of primary and tertiary alcohol) (1 g, 3.5 mmol) was dissolved in dry pyridine (10 ml) in a flask under an atmosphere of nitrogen and the solution was cooled to 0° C. (ice bath). To this was added dropwise Tosyl chloride (0.333 g, 1.75 mmol, 0.5 eq.) in solution in pyridine (2 ml). The resulting mixture was stirred in the ice bath for 2 h then allowed to reach room temperature O/N. The reaction was monitored by TLC/LCMS. And an other aliquot of TsCl (100 mg) was added and the reaction was stirred at rt for another 24 h at which point the reaction is considered completed. The pyridine is evaporated and the crude wax is re-dissolved in AcOEt before washing with 2N HCl. The aqueous phase was washed once more with AcOEt. The combined organic layers were washed with saturated solution of NH₄Cl and NaHCO₃, finally with bane, dried over Na₂SO₄ filtered and evaporated. The crude was purified by column chromatography (silica 50 g) using a gradient Pet Ether to 100% DCM. Then flush with AcOEt (to 100%). The fractions were analysed by TLC (DCM Rf TM 0.7, PMA). The tubes with TM were combined and evaporated to give a colourless wax (237 mg, 31%).

NMR analysis of the recovered alcohol (65%) shows it is still a mix of primary with enriched tertiary alcohol. The mix can be recycled for another tosylation cycle.

### 6-(benzyloxy)-2,2-dimethyl-2,3-dihydrobenzo[b][1,4]dioxine (11.5)

The titled compound was prepared according to the general procedure described in http://pubs.acs.org/doi/abs/10.1021/jo00880a045 (J. Org. Chem., 1976, 41 (18), pp 3064-3066) with tosylate (11.4) (367 mg, 0.83 mmol, 1 eq.) to yield to (11.5) as a colourless oil which solidifies upon standing at rt (m=173 mg, 77%) after purification (silica, gradient Pet Ether/DCM/AcOEt).

### 2,2-dimethyl-2,3-dihydrobenzo[b][1.4]dioxin-6-ol (11.6)

The titled compound was prepared by hydrogenation (with Pd/C in AcOEt) of (11.5) (173 mg, 0.63 mmol) to afford, after filtration on silica, (11.6) (107 mg. 97%) as a pale yellow oil.

### (S)-2,2-dimethyl-6-(oxiran-2-ylmethoxy)-2,3-dihydrobenzo[b][1,4]dioxine (11.7)

The titled compound was prepared according to the general procedure described in Sharpless and Al, JOC, 54(6), 1989, 1295-1304, (11.6) (105 mg, 0.58 mmol, 1 eq.) to yield to (11.7) as a colourless oil which solidifies upon standing at rt (m=119 mg, 86%) after purification (TLC (DCM/PMA)) (silica, gradient Pet Ether DCM/AcOEt 50/50 to 100% then 20% AcOEt).

### (S)-4-(2-(3-(2,2-dimethyl-2,3-dihydrobenzo[b][1,4]dioxin-6-yloxy)-2-hydroxypropyl-amino)ethoxy)benzamide (11.8)

### Example 44

Example 44 was prepared by coupling epoxide (11.6) ((60 mg, 0.25 mmol) and amine (6.10) (3 eq.), K₂CO₃ (3.1 eq.) in HFIP/water (4/1) by the method (ii) described below to afford Ex 44 as a white solid (56 mg, 53%).

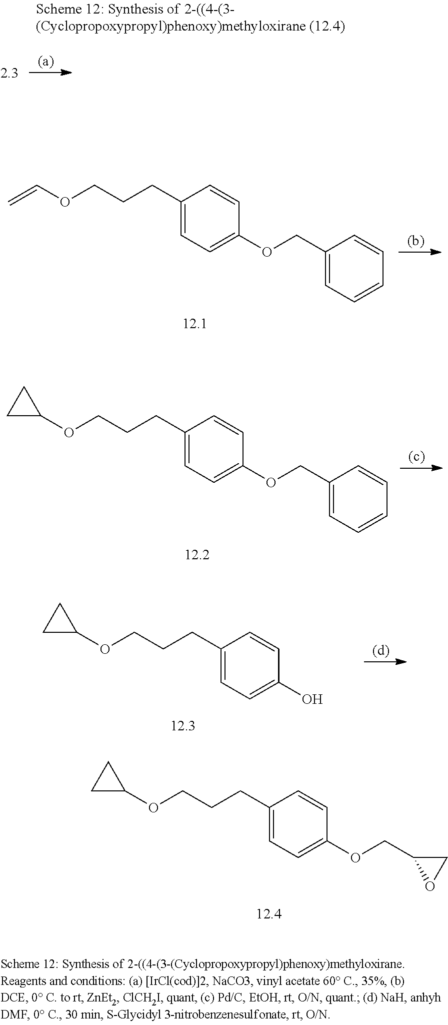 [SMILES: C=COCCCc1ccc(OCc2ccccc2)cc1 ; c1ccc(COc2ccc(CCCOC3CC3)cc2)cc1 ; Oc1ccc(CCCOC2CC2)cc1 ; c1cc(OC[C@@H]2CO2)ccc1CCCOC1CC1]

The synthesis of (12.4) was realised from (2.3) as outlined in scheme 12.

Step (a): The preparation of vinyl ether (12.1) was adapted from the experimental described in patent number WO2009/026537 A1
Step (b): The Simmons Smith reaction on (121) was adapted from S. E. Denmark et al., J. Org, Chem., 1991, 56 (25), 6974-6981 (and ref cited) to gives (12.2).
Step (c): Hydrogenation (with Pd/C in EtOH) of (12.2) gives (12.3) in quantitative yield.
Step (d): Synthesis of oxirane (12.4) was adapted from Eur, J, Med. Chem. 37, 2002, 731-741, using (S)-Glycidyl 3-nitrobenzenesulfonate.

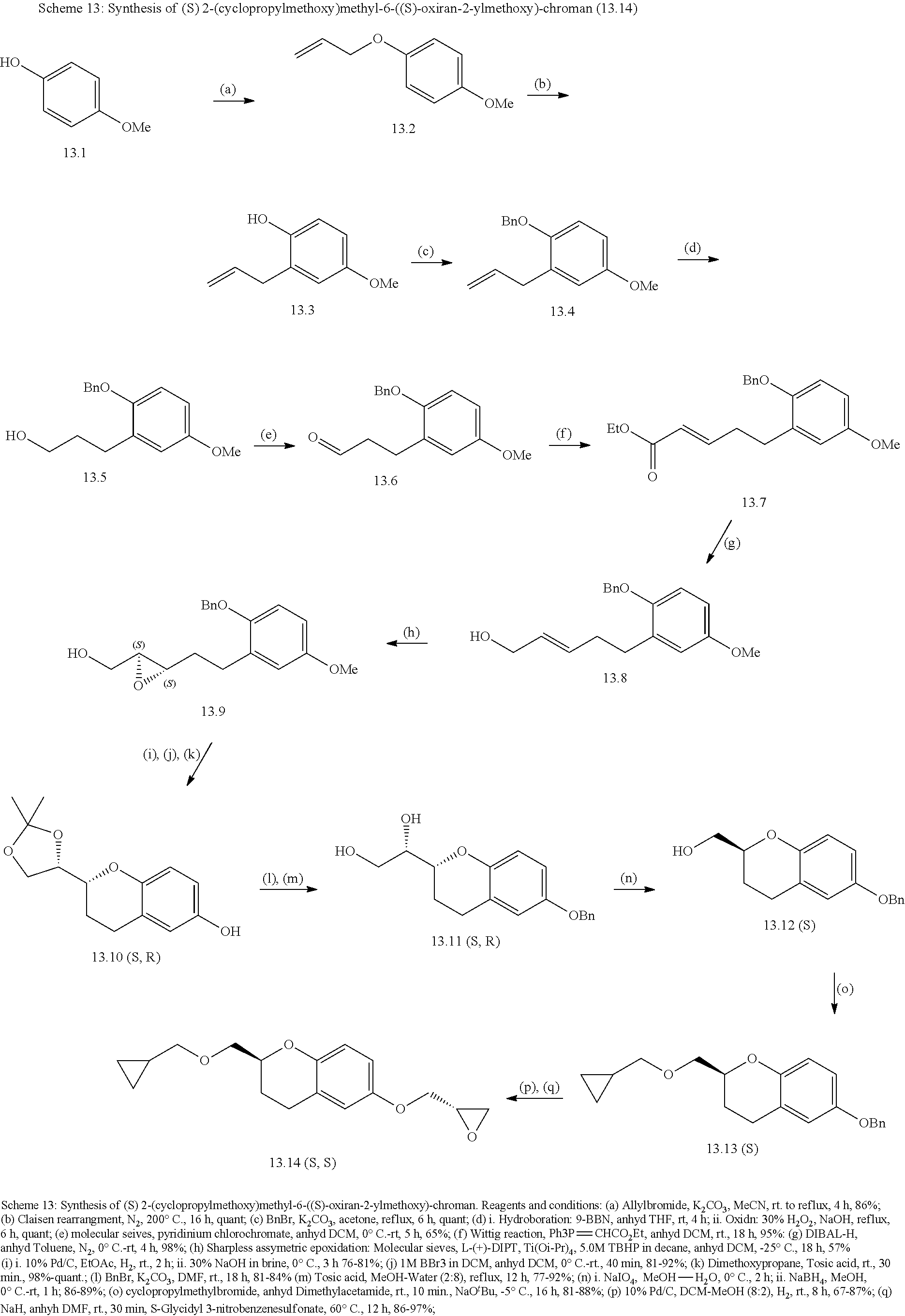

### Step (a): 1-(allyloxy)-4-methoxybenzene (13.2)

To a stirred solution of 4-methoxyphenol (13.1) (50 g, 402.8 mmol) in acetonitrile (500 mL) were added K₂CO₃ (61.23 g, 443 mmol) and allyl bromide (38.34 mL, 443 mmol) at room temperature, and stirring was continued for 4 h under reflux. After cooling to rt, filtration of the reaction mixture followed by concentration, the residue was partitioned between water (200 mL) and ether (3×300 mL). Combined ether layers were washed with a solution of 2M NaOH (1×250 mL), water (1×200 mL), brine (1×200 mL) and dried over Na₂SO₄ and concentrated to afford the title product (13.2) as yellow oil (86%).

### Step (b): 2-allyl-4-methoxyphenol (13.3)

(Tetrahedron 62 (2006) 5883-5896). Neat allyl phenyl ether (13.2) (57 g, 34.7 mmol) was placed in an oven dried flask or a sealed tube and heated at 200° C. under a nitrogen atmosphere for 16 h. The reaction mixture changed from colourless oil to pale brown oil. The reaction mixture was allowed to cool and the thick oil was passed through a silica plug using a gradient of ethyl acetate in Pet Ether to afford (13.3) (quantitative yield).

The synthesis from Step (c) (13.3) to Step (n) (13.12) was adapted from Synthesis, 2009, 11, 1886-1896.

### Step (k): (R)-2-((S)-2,2-dimethyl-1,3-dioxolan-4-yl)chroman-6-ol (13.10)

(S)-1-((R)-6-hydroxychroman-2-0)ethane-1,2-diol (from Step (j)) (2 mmol) and dimethoxypropane (5.0 mL) were stirred with p-toluenesulfonic acid (10 mol %; 0.2 mmol) for 30 min at rt. And a saturated aqueous solution of NaHCO₂, was added. The aqueous layer was extracted with ethyl acetate (3×10 mL). The combined organic phases were washed with brine, dried over Na₂SO₄ and the solvent evaporated. (13.10) (99%) was purified as yellow oil by FCC on silica using a gradient of up to 15% EtOAc in Pet ether.

### Step (l): (R)-(6-benzyloxy)-2-((S)-2,2-dimethyl-1,3-dioxolan-4-0)chroman

To a stirred solution of phenol (13.10) (2 mmol) in dry DMF (10 mL) was added K₂CO₃ (5 mmol, 2.5 eq) and the mixture was stirred at rt for 30 min. To this was added Benzyl bromide (2.2 mmol, 1.1 eq) and stirring was continued overnight. Reaction was monitored by LCMS. DMF was removed under high vacuum and the residue was partitioned between diethylether (40 mL) and water (10 mL). The organic layer was again washed with water (1×20 mL) and brine (1×25 mL). After drying over Na₂SO₄ and filtration, solvent was removed by rotary evaporation to afford the desired benzylated product as white solids. These compounds were taken forward without further purification

### Step (m): (S)-1-((R)-6-benzyloxychroman-2-0)ethane-1,2-diol (13.11)

The product obtained from step (I) (1.5 mmol) was dissolved in methanol (5 mL) and water (10 mL). To this was added TsOH (2.25 mmol, 1.5 eq) and the solution was refluxed for 12 h. Completion of the reaction was monitored by TLC in Pet Ether/EtOAc (1:1). The solution was concentrated and some more water (10 mL) was added. The product was extracted in EtOAc (2×20 mL). The organic layers were combined, washed with NaHCO₃ (2×20 mL), brine (20 mL) and dried over Na₂SO₄, Solvents were evaporated to afford the desired product (13.11) (92%) as white solids,

### Step (n): (S)-(6-benzyloxychroman-2-yl)methanol (13.12)

(i): To a stirred solution of diol (13.11) (1 mmol) in MeOH (8 mL) at 0° C. was added a solution of NaIO₄ (1.6 mmol) in H₂O (4 mL). After stirring the reaction mixture at O′ t for 2 h, MeOH was evaporated in vacuo at low temperature. The aqueous solution was extracted with DCM (2×10 mL). The combined organic layers were further washed with H₂O (10 mL), brine (10 mL), dried over Na₂SO₄, filtered and concentrated under reduced pressure to yield the crude aldehyde as a colourless gum, which was used without further purification.
(ii): To an ice-cooled solution of the crude aldehyde (1 mmol) in MeOH (6 mL) was added NaBH₄ (1.2 mmol) and the mixture was stirred for 30 min at 0° C. MeOH was removed under reduced pressure. Residue was re-dissolved in EtOAc (30 mL) and the organic layer was washed with water (2×10 mL), brine (15 mL) and dried over Na₂SO₄. EtOAc was removed by rotary evaporator to afford the desired product (13.12) (86%) as off white solids

### Step (o): (S)-(6-benzyloxy)-2-((cyclopropylmethoxy)methyl)chroman (13.13)

The Synthesis of (13.13) was adapted from Tetrahedron, 2007, 63, 1872-1876.

### Step (p) Catalytic Hydrogenation

To a solution of (13.13) in DCM/MeOH (8:2) was added 10% w/w Pd/C. (When the amount of Pd/C required was more than 1 g, the Pd/C is added as a suspension in water (1-2 mL)). This suspension was degassed, placed under a hydrogen atmosphere and was stirred for 12 h at rt. Completion of the reaction was monitored by LCMS or TLC. Upon completion, the suspension was filtered through a bed of celite with some DCM washing. The organic phase was concentrated to get oil and the residue was then partitioned between DCM and water. The organic layer was washed with brine, dried over sodium sulphate and the DCM was evaporated to give a brownish oil. The oil was purified by passing it through a plug of silica and using Pet ether/EtOAc (7:3) solvent system to give a sticky colourless solid,

### Step (q): (S)-2-((cyclopropylmethoxy)methyl)-6-((S)-oxiran-2-ylmethoxy)chroman (13.14)

Synthesis of oxiran (13.14) was adapted from Eur. J. Med. Chem., 37, 2002, 731-741. (13.14) (87%) was obtained as pale orange oil and was used without further purification.

Examples 1-29 and 45-46 and Example 30 to 44 were Prepared as Outlined in Schemes 9-11

Examples 1-29 and 45-46 were prepared by coupling the intermediates described above (epoxides and amine (as a free base or salts)) by the methods (i) or (ii) as described below: Examples 30-44 were prepared as described in schemes 9-10-11.

### Typical Procedures for Epoxide Opening:

(i) In a microwave vessel, the corresponding glycidyl ether (1 eq.) and corresponding amine salt (1.5 or 2 eq) is dissolved in a mixture iPrOH/MeCN/water (7:2:1), followed by triethylamine or diisopropylethylamine (1.1 eq. to the amine salt stoechiometry). The vessel is then heated for 60 min. at 90° C. in the MW reactor.

The crude is then transferred into a round bottom flask; Isolute® is added to the solution and evaporated to dryness under reduced pressure before purification by FCC using an eluting gradient of 1N NH₃ in MeOH and DCM.

### (ii) Alternative Solvent Mix and Base:

Following the same stoechiometries and protocol, the solvent in (i) can be substituted with a mixture of Hexafluoroisopropanal/water (4/1) and the triethylamine/DIPEA with an inorganic base such as K₂CO₃, for example. The base substitution avoids contamination from the tertiary amine (Et₃N or DIPEA) in the final compound.

In the case of free amine, the tertiary amine base or inorganic base is omitted.

DATA TABLE 1
Analytical data for analogues of aryloxypropanolamine

| Example | mp/° C. | LCMS/(M + 1)⁺/z | stereo | Salt |
|---|---|---|---|---|
| 1 | N/A | C18: 2′06/459.2; | rac. | |
| | | Luna: 2′21 | | |
| 2 | Wax | C18: 2′26/435.7; | rac. | |
| | | Luna: 2′47 | | |
| 3 | Wax | C18: 2′26/446.5; | rac. | |
| | | Luna: 2′48 | | |
| 4 | 175-178 | C18: 2′11/485.7; | (S) | |
| | | Luna: 2′25 | | |
| 5 | 123-125 | C18: 2′11/474.6; | (S) | |
| | | Luna: 2′25 | | |
| 6 | 73-75 | C18: 2′17/487.5; | (S) | |
| | | Luna: 2′32 | | |
| 7 | 121.5-124 | C18: 2′18/488.6; | (S) | |
| | | Luna: 2′34 | | |
| 8 | 132-135 | C18: 2′10/443.4; | (S) | |
| | | Luna: 2′24 | | |
| 9 | 120-122 | C18: 2′18/457.7; | (S) | |
| | | Luna: 2′34 | | |
| 10 | 170-175 | C18: 2′05/490.6; | (R,S) | |
| | | Luna: 2′22 | | |
| 11 | 136-138 | C18: 2′03/473.3; | (R,S) | |
| | | Luna: 2′17 | | |
| 12 | 128-130 | C18: 1′99/451.7; | (S) | |
| | | Luna: 2′10 | | |
| 13 | 105-108 | C18: 2′15/458.1; | (S) | |
| | | Luna: 2′26 | | |
| 14 | N/A | C18: 1′89/481.5; | (R,S) | |
| | | Luna: 2′08 | | |
| 15 | 123-125 | C18: 2′08/487.5; | (R,S) | |
| | | Luna: 2′25 | | |
| 16 | 134-136 | C18: 2′13/487.7; | (R,S) | |
| | | Luna: 2′28 | | |
| 17 | 157-159 | C18: 2′09/456.2; | (S) | |
| | | Luna: 2′21 | | |
| 18 | 159-162 | C18: 2′01/485.4; | (R,S) | |
| | | Luna: 2′18 | | |
| 19 | 144-146 | C18: 2′05/472.2; | (S,S) | |
| | | Luna: 2′19 | | |
| 20 | 126-129 | C18: 2′02/430.0; | (S) | |
| | | Luna: 2′14 | | |
| 21 | N/A | C18: 2′15/452.4; | (S) | |
| | | Luna: 2′34 | | |
| 22 | 183-185 | C18: 2′08/456.2; | (S) | |
| | | Luna: 2′23 | | |
| 23 | N/A | C18: 2′09/483.3; | (R,S) | |
| | | Luna: 2′24 | | |
| 24 | 128-130 | C18: 2′02/472.2; | (R,S) | |
| | | Luna: 2′17 | | |
| 25 | N/A | C18: 2′19/525.3; | (R,S) | |
| | | Luna: 2′42 | | |
| 26 | 161-164 | C18: 2′20/525.2; | (S,S) | |
| | | Luna: 2′45 | | |
| 27 | 263-266 | C18: 2′21/452.4; | (S) | |
| | | Luna: 2′40 | | |
| 28 | 148-150 | C18: 2′17/465.2; | (S) | |
| | | Luna: 2′36 | | |
| 29 | 194-196 | C18: 2′19/463.9; | (S) | |
| | | Luna: 2′40 | | |
| 30 | 192-194 | C18: 0′43/508.2; | (R,S) | |
| | | Luna: 0′67 | | |
| 31 | Decomp. 245 | C18: 2′20/538.3; | (R,S) | |
| | | Luna: 2′38 | | |
| 32 | Decomp. 205 | C18: 2′08/508.3; | (S,S) | AcOH |
| | | Luna: 2′24 | | |
| 33 | Decomp. 195 | C18: 2′20/525.9; | (R,S) | AcOH |
| | | Luna: 2′42 | | |
| 34 | 188-189 | C18: 2′34/591.9; | (R,S) | |
| | | Luna: 2′58 | | |
| 35 | 162-164 | C18: 2′20/525.4; | (R,S) | |
| | | Luna: 2′40 | | |
| 36 | 149-152 | C18: 2′13/533.5; | (R,S) | |
| | | Luna: 2′35 | | |
| 37 | 182-184 | C18: 2′26/573.1; | (R,S) | |
| | | Luna: 2′49 | | |
| 38 | 150-151 | C18: 2′20/544.1; | (R,S) | |
| | | Luna: 2′42 | | |
| 39 | 146-148 | C18: 2′27/520.9; | (R,S) | |
| | | Luna: 2′41 | | |
| 40 | 145-147 | C18: 2′22/555.0; | (R,S) | |
| | | Luna: 2′41 | | |
| 41 | 234-235 | C18: 0′41/492.3; | (S) | HCl |
| | | Luna: 0′59 | | |
| 42 | 132-133 | C18: 0′40/504.4; | (S) | |
| | | Luna: 0′63 | | |
| 43 | 134-135 | C18: 0′42/534.3; | (R,S) | |
| | | Luna: 0′74 | | |
| 44 | 101-103 | C18: 1′95/417.9; | (S) | |
| | | Luna: 2′13 | | |
| 45 | 158-159 | C18: 2′10/442.0; | (S) | |
| | | Luna: 2′22 | | |
| 46 | 126-128 | C18: 2′08/429.9; | (S) | |
| | | Luna: 2′23 | | |

### Example A1

### Ligand Binding Studies

Selectivity of ligands for the three beta-adrenoceptors was assessed by whole-cell binding studies using ³H-CGP12177 in CHO cells expressing the human beta1, beta2 or beta3-adrenoceptors respectively essentially as described by Baker (2005; Br. J Pharmacol: 144, 317-22) Values shown are K_{D} values determined as described by Baker (2005). The K₀ values for each ligand at the human beta 1 and beta 2 adrenoceptors are shown in Table 3. K_{D} represents the concentration of compound required to occupy 50% of the receptors in cells or tissues.

The selectivity of a ligand is given by the ratio of beta-1 to beta-2 K_{D}. Accordingly a difference of one in the logarithmic values thereof represents a 10-fold selectivity, a difference of 2 represents 100-fold selectivity and a difference of 3 represents 1000-fold selectivity etc.

TABLE 3
3H-CGP 12177 Whole cell binding

| | Beta 1 | Beta 2 | Beta 1-Beta 2 |
|---|---|---|---|
| Ex No | Log K_{D} | Log K_{D} | Log K_{D} |
| 1 | −8.48 | −6.07 | 2.40 |
| 2 | −8.05 | −6.04 | 2.01 |
| 3 | −8.13 | −6.12 | 2.01 |
| 4 | −8.36 | −6.46 | 1.91 |
| 5 | −8.81 | −6.34 | 2.47 |
| 6 | −7.94 | −5.98 | 1.96 |
| 7 | −8.62 | −6.57 | 2.06 |
| 8 | −8.54 | −5.88 | 2.66 |
| 9 | −9.04 | −6.45 | 2.59 |
| 10 | −9.01 | −6.87 | 2.14 |
| 11 | −7.63 | −4.99 | 2.64 |
| 12 | −9.08 | 6.86 | 2.22 |
| 13 | −8.83 | −6.53 | 2.3 |
| 14 | −8.38 | −5.90 | 2.48 |
| 15 | −8.12 | −5.36 | 2.76 |
| 16 | −8.21 | −5.30 | 2.91 |
| 17 | −9.15 | −6.52 | 2.63 |
| 18 | −8.34 | −5.33 | 3.01 |
| 19 | −8.15 | −5.82 | 2.33 |
| 20 | −8.99 | −6.71 | 2.28 |
| 21 | −8.56 | −6.37 | 2.19 |
| 22 | −8.08 | −5.85 | 2.23 |
| 23 | −8.05 | −5.58 | 2.47 |
| 24 | −8.57 | −6.17 | 2.4 |
| 25 | −9.10 | −6.46 | 2.64 |
| 26 | −8.38 | −5.60 | 2.78 |
| 27 | −6.87 | >−4.00 | <2.87 |
| 28 | −6.93 | >−4.00 | <2.93 |
| 29 | −7.10 | >−4.00 | <3.10 |
| 30 | −7.24 | −4.14 | 3.10 |
| 31 | −8.11 | −5.10 | 3.01 |
| 31 | −7.71 | −4.95 | 2.76 |
| 33 | −8.55 | −5.70 | 2.85 |
| 34 | −8.47 | −5.11 | 3.37 |
| 35 | −8.68 | −4.98 | 3.70 |
| 36 | −8.06 | −5.40 | 2.66 |
| 37 | −8.57 | −5.20 | 3.37 |
| 38 | −8.54 | −5.20 | 3.34 |
| 39 | −8.97 | −6.06 | 2.91 |
| 40 | −8.49 | −5.79 | 2.70 |
| 41 | −8.74 | −5.87 | 2.87 |
| 42 | −8.71 | −6.39 | 2.32 |
| 43 | −8.37 | −5.56 | 2.81 |
| 44 | −6.50 | >−4.00 | <2.50 |
| 45 | −9.17 | −6.52 | 2.65 |
| 46 | −8.67 | −6.10 | 2.57 |

1. A compound of Formula (I), and its pharmaceutically acceptable salt or salts and physiologically hydrolysable derivatives in free form or salt form: 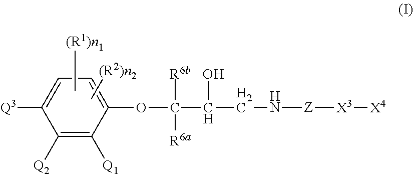
wherein
R¹ is independently selected from F, Cl, Br, CN, NH₂, OH, CHO, COOH, oxo, C₁₋₄alkyl, C₁₋₄alkoxy, CONH₂ (optionally mono- or di-substituted by C₁₋₄alkyl) and SO₂NH₂,
R² is independently selected from C₁₋₆allkyl substituted by R³ wherein the C₁₋₆alkyl chain optionally comprises one or two heteroatoms select from O;
R³ is selected from aryl, C₃₋₆cycloalkyl, C₃₋₆heterocyclyl and C₃₋₆heteroaryl, wherein the heterocyclyl and heteroaryl rings are nitrogen containing;
and wherein R³ is optonally substituted by one or more groups selected from R¹;
n1 is zero or an integer from 1 to 2;
n2 is an integer from 1 to 2;
and the sum of n1 and 2 is less than or equal to 2;
R⁵ is selected from any group defined for R¹ and R²;
R^{6a} and R^{6b} are independently selected from H or C₁₋₄alkyl;
R⁷ is independently selected from F, Cl, Br, CN, NH₂, OH, CHO COOH, oxo, C₁₋₄alkoxy, C₁₋₄alkoxy, CONH₂ (optionally mono- or di-substituted by C₁₋₄alkyl) and SO₂NH₂,
Q¹, Q² and Q³ are independently selected from H or any group defined for R¹ and R²;
or
Q¹ and Q² or Q² and Q³ together form a C₅₋₆heteroaryl or C₅₋₆heterocylclic ring; optionally containing one or two heteroatoms selected from N and O
optionally substituted by upto two groups selected from R⁵;
Z is selected from linear C₂₋₃ alkylene;
X³ is O;
X⁴ is selected from aryl, a 9-10 membered heteroaryl ring or a 9-10 membered heterocyclic ring, wherein the heteroaryl and heterocyclic rings contain one or more heteroatoms selected from N, and optionally additionally O,
and wherein X⁴ is optionally substituted by one or two oxo moieties and is optionally substituted by one or more groups selected from R⁷;
with the proviso that:
(i) when X⁴ is phenyl then Q¹ and Q² or Q² and Q³—together form an optionally substituted heteroaryl or heterocylclic ring as defined above; and
(ii) when Q¹, Q² and Q³ are independently selected from H or any group defined for R¹ and R² then X⁴ is not phenyl except when R² is C₁₋₅alkyl substituted by R³ wherein R³ is C₃₋₆heterocyclyl as defined above.

2. The compound as claimed in claim 1, wherein Q¹ and Q² or Q² and Q³ together form a C₅₋₆heteroaryl or C₅₋₆heterocylclic ring; optionally containing one or two heteroatoms selected from N and O, optionally substituted by up to two groups selected from R⁵.

3. The compound as claimed in claim 2, wherein

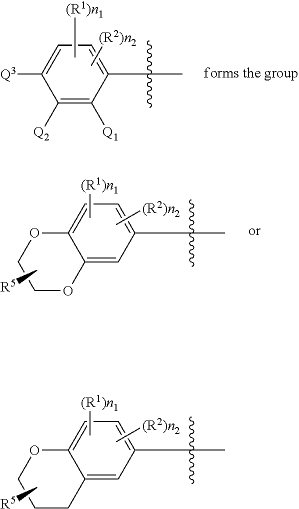

4. The compound as claimed in claim 1, wherein R¹ is chloro, bromo or fluoro, C₁₋₄alkyl, C₁₋₄alkoxy or cyano.

5. The compound as claimed in claim 1, wherein R² is C₃₋₆cycloalkylC₁₋₅alkyl or phenylC₁₋₅alkyl where the C₁₋₅alkyl optionally contains 1 or 2 heteroatoms selected from O.

6. The compound as claimed in claim 1, wherein R^{6a} and R^{6b} are both hydrogen.

7. The compound as claimed in claim 1, wherein X³ is —O—.

8. The compound as claimed in claim 1, wherein X⁴ is phenyl or a 9-10 membered heteroaryl ring.

9. The compound as claimed in claim 1, wherein X⁴ is a 9-10 membered heterocyclic ring selected from isoindoline and 2,3-dihydroxybenzimidazole

10. The compound as claimed in claim 1, wherein and R⁷ is selected from amino, carboxy, halo, C₁₋₄alkyl, C₁₋₄alkoxy, C₁₋₂perfluoroalkyl, oxo, —NHC(O)C₁₋₄alkyl or —CONH₂.

11. The compound as claimed in claim 1, wherein the compound is selected from a compound of Formula (I) according to any one of Examples 1-46 as shown in Table 1:
TABLE 1
Description of compounds of formula II wherein n1 and n2 are both 0:

| | |
|---|---|
| | (II) |
| 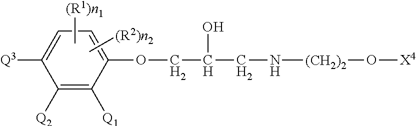 | |
| Ex | Q¹ | Q² | Q³ | 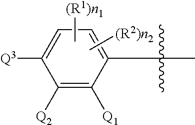 | R⁵ | X4 | R⁷, R⁸ | enanti- omer |
|---|---|---|---|---|---|---|---|---|
| 1 | H | H | 4-(2- | | | Ph | 4-CONH₂ | rac. |
| | | | (c. pentyloxy) | | | | | |
| | | | ethoxy) | | | | | |
| 2 | H | H | 4-(2- | | | Ph | 4-F | rac. |
| | | | (c. pentyloxy) | | | | | |
| | | | ethoxy) | | | | | |
| 3 | H | H | 4-(2- | | | Ph | 4-OMe | rac. |
| | | | (c. pentyloxy) | | | | | |
| | | | ethoxy) | | | | | |
| 4 | H | H | 4-(2- | | | 6- | 2-oxo | (S) |
| | | | (c. pentyloxy) | | | dih- | | |
| | | | ethoxy) | | | iQn | | |
| 5 | H | H | 4-(2- | | | Ph | 4- | (S) |
| | | | (c. pentyloxy) | | | | CONHMe | |
| | | | ethoxy) | | | | | |
| 6 | H | H | 4-(2- | | | Ph | 4- | (S) |
| | | | (c. pentyloxy) | | | | CONMe₂ | |
| | | | ethoxy) | | | | | |
| 7 | H | H | 4-(2- | | | Ph | 4- | (S) |
| | | | (c. pentyloxy) | | | | CONHEt | |
| | | | ethoxy) | | | | | |
| 8 | H | H | 4- | — | — | Ph | 4-CONH₂ | (S) |
| | | | c. prCH₂O(CH₂)₃ | | | | | |
| 9 | H | H | 4- | — | — | Ph | 4-CONH₂ | (S) |
| | | | c. pentO(CH₂)₃ | | | | | |
| 10 | | | — | 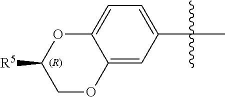 | c. prCH₂OCH₂ | Ph | 3-CONH₂ 4-OH | (R, S) |
| 11 | | | — | 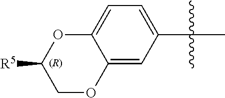 | c. prCH₂OCH₂ | Ph | 4-CONH₂ | (R, S) |
| 12 | H | H | 4- | — | — | 6-Qnl | — | (S) |
| | | | c. prCH₂O(CH₂)₃ | | | | | |
| 13 | H | H | 4- | — | — | Ph | 4- | (S) |
| | | | c. prCH₂O(CH₂)₃ | | | | NHCOCH₃ | |
| 14 | | | — | 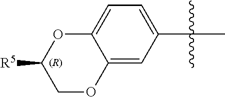 | c. prCH₂OCH₂ | 6-Qnl | — | (R, S) |
| 15 | | | — | 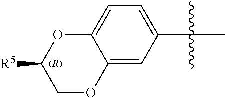 | c. prCH₂OCH₂ | Ph | 4- NHCOCH₃ | (R, S) |
| 16 | | | — | 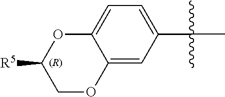 | c. pentOCH₂ | Ph | 4-CONH₂ | (R, S) |
| 17 | H | H | 4- | — | — | 5-i- | 1-oxo | (S) |
| | | | c. prCH₂O(CH₂)₃ | | | indl | | |
| 18 | | | | 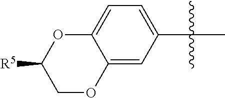 | c. prCH₂OCH₂ | 5-i- indl | 1-oxo | (R, S) |
| 19 | | | — | 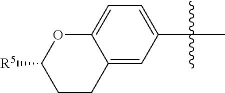 | c. prCH₂OCH₂ | Ph | 4-CONH₂ | (S, S) |
| 20 | H | H | 4- | — | — | Ph | 4-CONH₂ | (S) |
| | | | c. prCH₂O(CH₂)₂ | | | | | |
| 21 | H | H | 4- | — | — | 6- | — | (S) |
| | | | c. prCH₂O(CH₂)₃ | | | Qox | | |
| 22 | H | H | 4- | — | — | 5- | 2-oxo | (S) |
| | | | c. prCH₂O(CH₂)₃ | | | dih- | | |
| | | | | | | bzl | | |
| 23 | | | — | 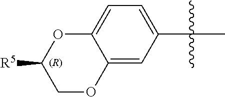 | c. prCH₂OCH₂ | 6- Qox | — | (R, S) |
| 24 | | | — | 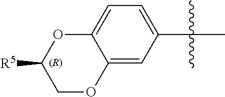 | c. prCH₂OCH₂ | 5- BzT | — | (R, S) |
| 25 | | | | 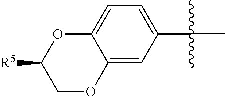 | 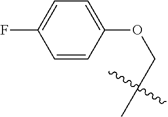 [SMILES: *COc1ccc(F)cc1] | 5-i- indl | 1-oxo | (R, S) |
| 26 | | | | 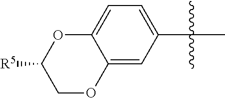 | 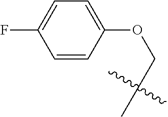 [SMILES: *COc1ccc(F)cc1] | 5-i- indl | 1-oxo | (S, S) |
| 27 | | | c. prCH₂O | 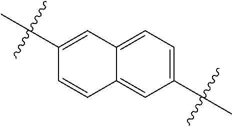 [SMILES: *c1ccc2cc(*)ccc2c1] | | Ph | 4-CONH₂ | (S) |
| 28 | | | c. prCH₂OCH₂ | 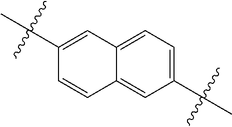 [SMILES: *c1ccc2cc(*)ccc2c1] | | Ph | 4-CONH₂ | (S) |
| 29 | | | c. prCH₂O | 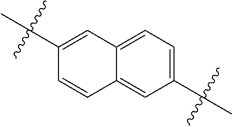 [SMILES: *c1ccc2cc(*)ccc2c1] | | 5-i- indl | 1-oxo | (S) |
| 30 | | | | 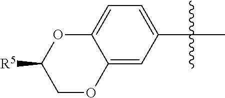 | 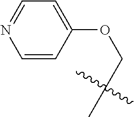 [SMILES: *COc1ccncc1] | 5-i- indl | 1-oxo | (R, S) |
| 31 | | | | 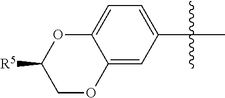 | 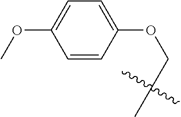 [SMILES: *COc1ccc(OC)cc1] | 5-i- indl | 1-oxo | (R, S) |
| 32 | | | | 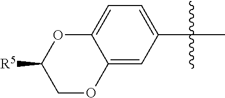 | 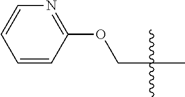 [SMILES: *COc1ccccn1] | 5-i- indl | 1-oxo | (R, S) |
| 33 | | | | 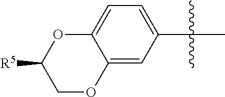 | 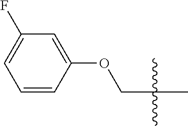 [SMILES: *COc1cccc(F)c1] | 5-i- indl | 1-oxo | (R, S) |
| 34 | | | | 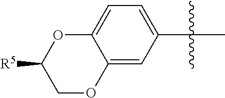 | 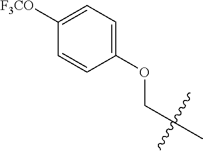 [SMILES: *COc1ccc(OC(F)(F)F)cc1] | 5-i- indl | 1-oxo | (R, S) |
| 35 | | | | 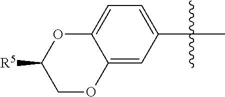 | 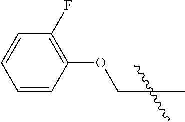 [SMILES: *COc1ccccc1F] | 5-i- indl | 1-oxo | (R, S) |
| 36 | | | | 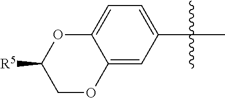 | 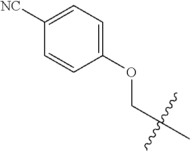 [SMILES: *COc1ccc(C#N)cc1] | 5-i- indl | 1-oxo | (R, S) |
| 37 | | | | 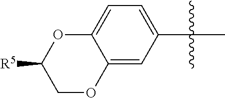 | 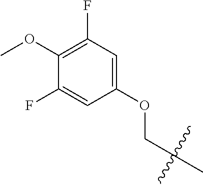 [SMILES: *COc1cc(F)c(OC)c(F)c1] | 5-i- indl | 1-oxo | (R, S) |
| 38 | | | | 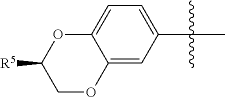 | 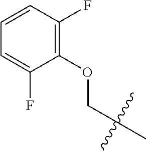 [SMILES: *COc1c(F)cccc1F] | 5-i- indl | 1-oxo | (R, S) |
| 39 | | | | 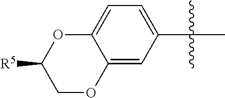 | 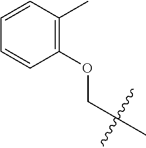 [SMILES: *COc1ccccc1C] | 5-i- indl | 1-oxo | (R, S) |
| 40 | | | | 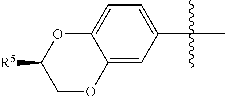 | 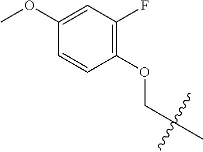 [SMILES: *COc1ccc(OC)cc1F] | 5-i- indl | 1-oxo | (R, S) |
| 41 | | | 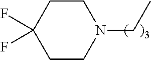 | | | Ph | 4-CONH₂ | (S) |
| 42 | | | 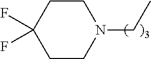 | | | 5-i- indl | 1-oxo | (S) |
| 43 | | | | 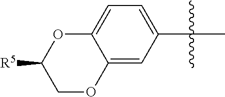 | 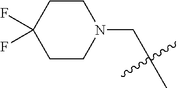 [SMILES: *CN1CCC(F)(F)CC1] | 5-i- indl | 1-oxo | (R, S) |
| 44 | | | | 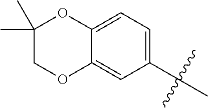 [SMILES: *c1ccc2c(c1)OCC(C)(C)O2] | | Ph | 4-CONH₂ | (S) |
| 45 | | | 4- | | | 5-i- | 1-oxo | (S) |
| | | | c. PrO(CH₂)₃ | | | indl | | |
| 46 | | | 4- | | | Ph | 4-CONH₂ | (S) |
| | | | c. PrO(CH₂)₃ | | | | | |

12. A composition comprising a therapeutically effective amount of a compound of Formula (I) or subformulae or its pharmaceutically acceptable salt and physiologically hydrolysable derivative as defined in claim 1 in association with one or more pharmaceutical carriers or diluents.

13. The compound as claimed in claim 1, for in the prevention or treatment of a condition selected from ischaemic heart disease, hypertension and heart failure, more preferably with concomitant respiratory disease, in particular asthma or COPD.

14. A process for the preparation of the compound of Formula (I) as claimed in claim 1, the process comprising a step selected from (a) to (e) as follows:
(a) Reaction of a compound of formula Pr1 with a compound of formula Pr2, 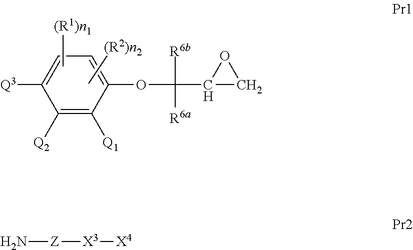
(b) Reaction of a compound of formula Pr3 with a compound of formula Pr4, 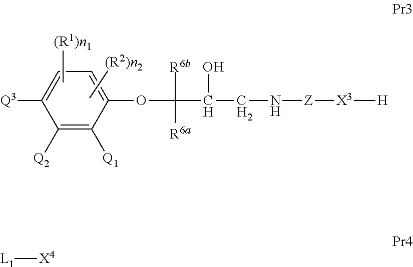
wherein L₁ is a leaving group;
(c) Reaction of a compound of formula Pr5 with a compound of formula Pr6, 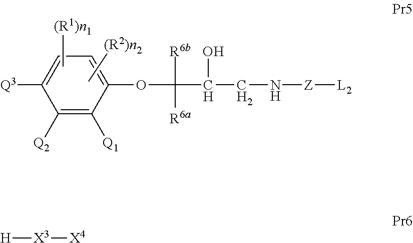
wherein L₂ is a leaving group;
(d) Reaction of a compound of formula Pr7 with a compound of formula Pr8; 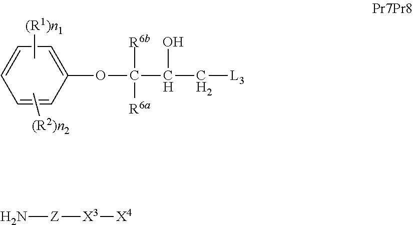
wherein L₃ is a leaving group
(e) Reaction of a compound of formula Pry with a compound of formula Pr6 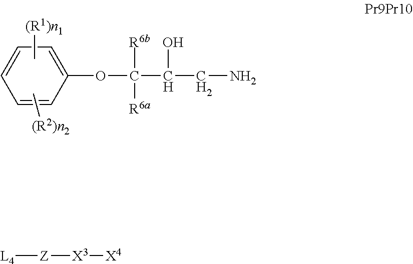
wherein L₄ is a leaving group
and thereafter if necessary:
(i) converting a compound of formula I into another compound of formula I;
(ii) removing any protecting groups; and/or
(iii) forming a salt, pro-drug or solvate.

15. A method for the treatment of a condition selected from ischaemic heart disease, hypertension and heart failure, more preferably with concomitant respiratory disease, in particular asthma or COPD, said method comprising administering to a subject in need thereof, a compound of formula I or subformulae or pharmaceutically acceptable salt or composition thereof as defined in claim 1, in an amount to treat the condition.
